(19) United States
(12) Patent Application Publication
(10) Pub. No.: US 2022/0283185 A1
(43) Pub. Date: Sep. 8, 2022

(54) ASSAYS FOR DETECTING AND QUANTIFYING A BIOMARKER OF PERICYTE INJURY

(71) Applicant: UNIVERSITY OF SOUTHERN CALIFORNIA, Los Angeles, CA (US)

(72) Inventors: Melanie D. Sweeney, Los Angeles, CA (US); Abhay P. Sagare, Los Angeles, CA (US); Berislav V. Zlokovic, Los Angeles, CA (US)

(21) Appl. No.: 17/637,700

(22) PCT Filed: Aug. 27, 2020
(86) PCT No.: PCT/US2020/048278

§ 371 Date: Feb. 23, 2022

Related U.S. Application Data
(60) Provisional application No. 62/892,195, filed on Aug. 27, 2019.

(51) Int. Cl. G01N 33/68; G01N 21/66; G01N 33/577; G01N 21/76

(52) U.S. Cl. CPC G01N 33/6896; G01N 21/66; G01N 33/577; G01N 21/76; G01N 2333/49; G01N 2800/2821

(57) ABSTRACT

A highly sensitive immunoassay has been developed and validated. In various embodiments, the assay comprises an immunoassay usable to measure soluble PDGFRβ (sPDGFR-β) in a human biofluid sample such as cerebrospinal fluid (CSF). In various embodiments, elevated sPDGFR-β in a human biofluid sample reflects pericyte and blood-brain barrier (BBB) injury, and is therefore an early biomarker of human cognitive dysfunction, dementia, and/or Alzheimer's disease.

### CROSS-REFERENCE TO RELATED APPLICATIONS

This Application claims the benefit of U.S. Provisional Patent Application No. 62/892,195, filed on Aug. 27, 2019, the disclosure of which is incorporated herein in its entirety by reference.

### STATEMENT REGARDING FEDERALLY SPONSORED RESEARCH

This invention was made with government support under National Institutes of Health (NIH) grants 5P01AG052350 and 5P50AG005142. The government has certain rights in the invention.

### FIELD

The present disclosure generally relates to certain medical and diagnostic assays, and, in particular, to an immunoassay capable of detecting a pericyte biomarker.

### BACKGROUND

Proper functioning of the central nervous system (CNS) requires highly coordinated actions of the neurovascular unit, which comprises vascular cells, glia, and neurons. Increasing evidence supports that cerebrovascular dysfunction contributes to complex neurodegenerative disorders, including Alzheimer's disease (AD). Human neuroimaging and biofluid studies have shown this during different stages of AD pathophysiology, as well as in a neuropathological analysis of AD brains. Multiple studies of AD brains reveal blood-brain barrier (BBB) breakdown with the accumulation of several blood-derived proteins in brain tissue and degeneration of brain capillary pericytes that is accelerated by apolipoprotein E ε4 (APOE4), the major genetic risk factor for sporadic AD.

Pericytes and vascular smooth muscle cells (SMCs) are vascular mural cells that tightly associate with the endothelium of brain capillaries and arteries/arterioles, respectively. Mural cell recruitment to the developing CNS vasculature is crucial for vascular angioarchitecture formation and stability, and this process is mediated via signaling events between endothelia-secreted platelet-derived growth factor (PDGF)-BB and PDGF receptor-β (PDGFRβ) expressed by mural cells. Both pericytes and SMCs highly express PDGFRβ during development, but PDGFRβ is predominately expressed by pericytes in the adult brain as reported in human tissue, human primary cells and rodent studies.

Pericytes are centrally positioned at the neurovascular unit (NVU) and are particularly vulnerable to injury and dysfunction that can disrupt BBB integrity and cerebral blood flow, causing proteins and other substances to release into the blood circulation. Pericyte injury results in cleavage of soluble PDGFRβ (sPDGFRβ) that is detectable in human and murine cerebrospinal fluid (CSF) and in serum and plasma portions of blood. Furthermore, CSF, serum and plasma sPDGFRβ levels are increased in humans during the early stages of cognitive impairment and positively correlate with hippocampal BBB breakdown in the aging human brain and in individuals with mild cognitive impairment, as shown by increased Kₜᵣₐₙₛ transfer constant values to gadolinium after dynamic contrast-enhanced magnetic resonance imaging. These studies support that BBB breakdown and pericyte injury measured by CSF, serum and plasma sPDGFRβ are early biomarkers of human cognitive dysfunction.

### SUMMARY

In an aspect of the present disclosure, an assay that identifies and quantifies sPDGFRβ in human biofluids, such as CSF, serum and plasma, is disclosed.

In various embodiments, the assay comprises an immunoassay capable of generating a detectable and measurable signal that correlates to the concentration of sPDGFRβ in the biofluid sample. The assay may comprise any type of colorimetric assay. For example, the detectable and measurable signal from the assay may comprise an absorbance, a fluorescence, or a luminescence, each consisting of any wavelength or range of wavelengths.

In various embodiments, the assay comprises a sandwich or self-sandwich immunoassay using the Meso Scale Discovery electrochemiluminescence (MSD-ECL) platform or other platform capable of quantitatively measuring a detection signal. In various embodiments, the assay comprises a self-sandwich assay where both the capture and detection antibodies comprise goat anti-human PDGFRβ polyclonal antibodies.

In various non-limiting embodiments, a study in accordance with the present disclosure screened combinations of five capture and three detecting antibodies and two human recombinant PDGFRβ proteins as standards on a Meso Scale Discovery electrochemiluminescence (MSD-ECL) platform to measure sPDGFRβ in human CSF from 147 individuals with normal cognition or early cognitive impairment.

In various embodiments, combinations of reagents, antibodies, and standards were used to identify and validate a self-sandwich immunoassay having inter- and intra-assay coefficient of variation <5%. Using this assay, elevated CSF sPDGFRβ levels in individuals with early cognitive impairment was confirmed, which supports the concept that sPDGFRβ is a promising and sensitive early biomarker of human cognitive dysfunction. The assay disclosed herein offers highly reproducible quantitative measurements of sPDGFRβ levels in human biofluids applicable at different clinicals sites. Moreover, the assay allows for future diagnostic and therapeutic studies of brain microvascular and BBB injury in different neurodegenerative disorders associated with neurovascular dysfunction and vascular contributions to cognitive impairment and dementia (VCID, sometimes referred to as “vascular dementia”).

The assay herein further provides indication of neurological disorders and BBB disruption in different CNS regions, such as in patients with Parkinson's Disease, Huntington's Disease, Human Immunodeficiency Virus (HIV)-dementia, Post-Traumatic Brain Syndrome, including post-Traumatic Brain Injury (TBI) related dementia (TBI-dem), small vessel disease of the brain, vascular dementia due to medical or environmental causes and/or any other type of CNS disorders associated with cognitive impairment and dementia.

In various embodiments of the present disclosure, a method for determining a concentration of soluble platelet-derived growth factor β (sPDGFRβ) in a biofluid sample from a human subject is provided; the method comprising forming a ternary complex of a detection antibody comprising a labelled anti-human PDGFRβ biotinylated antibody, sPDGFRβ present in the biofluid sample, and a capture antibody comprising an anti-human PDGFRβ antibody, wherein the anti-human PDGFRβ antibody is bound to a surface; detecting an intensity of light emission from the ternary complex; and interpolating the intensity of the light emission on a calibration curve to obtain the concentration of sPDGFRβ in the biofluid sample, wherein the labelled anti-human PDGFRβ biotinylated antibody comprises a conjugate between an immunoassay detection reagent and the anti-human PDGFRβ biotinylated antibody.

In various embodiments, the capture antibody comprises a goat anti-human PDGFRβ polyclonal antibody. In various embodiments, the detection antibody comprises a goat anti-human PDGFRβ biotinylated polyclonal antibody. In various embodiments, the biofluid comprises human cerebrospinal fluid (CSF), blood serum or blood plasma. In various embodiments, the concentration of sPDGFRβ in the biofluid sample is from about 100 pg/mL to about 30,000 pg/mL. In various embodiments, the immunoassay detection reagent comprises a sulfur-tagged streptavidin reagent. In various embodiments, the labelled anti-human PDGFRβ biotinylated antibody further comprises a streptavidin-biotin conjugated electrochemiluminescence label.

In various aspects, the method further comprises applying a voltage to the ternary complex during the detecting step. In various embodiments, the surface comprises an electrode surface disposed in a well plate. In various aspects, the detecting step further comprises detection of an electrochemiluminescence intensity upon insertion of the well plate into an imager having electrochemiluminescence detection. In various embodiments, the calibration curve comprises an x/y plot of electrochemiluminescence intensity versus sPDGFRβ concentration.

In various embodiments, the capture antibody is bound to a bottom of the well plate by spot-coating the bottom of the well plate with a phosphate buffered solution comprising a goat anti-human PDGFRβ polyclonal antibody and polysorbate 20. In various embodiments, the ternary complex is formed in a two-step process consisting of: (a) exposing the bound goat anti-human PDGFRβ polyclonal antibody in the well plate to a diluted aliquot of the biofluid sample to form a binary complex of sPDGFRβ and the capture antibody; and (b) exposing the binary complex to a solution comprising a labelled goat anti-human PDGFRβ biotinylated polyclonal antibody.

In various embodiments, the presence of sPDGFRβ in the biofluid sample provides a pericyte injury biomarker indicative of brain microvascular and blood brain barrier (BBB) injury. In various embodiments, the presence of sPDGFRβ in the biofluid sample indicates presence of at least one neurodegenerative disorder selected from Parkinson's Disease, Huntington's Disease, Human Immunodeficiency Virus (HIV)-dementia, or Post-Traumatic Brain Syndrome.

In various embodiments, the immunoassay detection reagent comprises horseradish peroxidase (HRP)-conjugated streptavidin. In various embodiments, the calibration curve comprises an x/y plot of absorbance versus sPDGFRβ concentration.

In various embodiments, a method of determining the presence of cognitive impairment or dementia in a human subject is provided; the method comprising obtaining a concentration of sPDGFRβ in a biofluid sample obtained from the human subject wherein the subject is categorized as having cognitive impairment or dementia if the sPDGFRβ in the biofluid sample is greater than about 4,000 pg/mL; wherein the concentration of sPDGFRβ in the biofluid sample is obtained by: forming a ternary complex of a detection antibody comprising a labelled anti-human PDGFRβ biotinylated antibody, sPDGFRβ present in the biofluid sample, and a capture antibody comprising an anti-human PDGFRβ antibody, wherein the anti-human PDGFRβ antibody is bound to a surface; detecting an intensity of light emission from the ternary complex; and interpolating the intensity of the light emission on a calibration curve to obtain the concentration of sPDGFRβ in the biofluid sample, wherein the labelled anti-human PDGFRβ biotinylated antibody comprises a conjugate between an immunoassay detection reagent and the anti-human PDGFRβ biotinylated antibody. In various embodiments, the human subject is categorized as having dementia if the sPDGFRβ in the biofluid sample from the subject is greater than about 5,000 pg/mL.

In various embodiments, a method of determining the presence of Alzheimer's disease in a human subject is provided; the method comprising obtaining a concentration of sPDGFRβ in a biofluid sample obtained from the human subject, wherein the subject is categorized as having Alzheimer's disease if the sPDGFRβ in the biofluid sample is greater than about 4,000 pg/mL; wherein the concentration of sPDGFRβ in the biofluid sample is obtained by: forming a ternary complex of a detection antibody comprising a labelled anti-human PDGFRβ biotinylated antibody, sPDGFRβ present in the biofluid sample, and a capture antibody comprising an anti-human PDGFRβ antibody, wherein the anti-human PDGFRβ antibody is bound to a surface; detecting an intensity of light emission from the ternary complex; and interpolating the intensity of the light emission on a calibration curve to obtain the concentration of sPDGFRβ in the biofluid sample, wherein the labelled anti-human PDGFRβ biotinylated antibody comprises a conjugate between an immunoassay detection reagent and the anti-human PDGFRβ biotinylated antibody. In various embodiments, the human subject is categorized as having Alzheimer's disease if the sPDGFRβ in the biofluid sample from the subject is greater than about 5,000 pg/mL.

In various embodiments, an assay system for determining a concentration of soluble platelet-derived growth factor R (sPDGFRβ) in a biofluid sample is provided; the assay system comprising: a ternary complex of a detection antibody comprising a labelled goat anti-human PDGFRβ biotinylated polyclonal antibody, sPDGFRβ present in the biofluid sample, and a capture antibody comprising a goat anti-human PDGFRβ polyclonal antibody, wherein the goat anti-human PDGFRβ polyclonal antibody is bound to a surface, and wherein the labelled goat anti-human PDGFRβ biotinylated antibody is a conjugation product of an immunoassay detection reagent and the goat anti-human PDGFRβ biotinylated polyclonal antibody.

### BRIEF DESCRIPTION OF THE FIGURES

The subject matter of the present disclosure is pointed out with particularity, and claimed distinctly in the concluding portion of the specification. A more complete understanding of the present disclosure, however, may best be obtained by referring to the detailed description and claims when considered in connection with the following drawing figures:

FIGS. 1a to 1d set forth the performance summary of the novel sPDGFRβ assay in accordance with the present disclosure. FIG. 1a illustrates representative standard curves plotting concentration and electrochemiluminescence signal of two recombinant standard proteins. FIG. 1b illustrates a dilution linearity test. FIG. 1c illustrates a parallelism test. FIG. 1d sets forth the summary of the sensitivity, linearity, and reproducibility of the assay.

FIGS. 2a to 2f set forth the validation of sPDGFRβ as a pericyte injury biomarker in human CSF. FIG. 2a illustrates the levels of CSF sPDGFRβ in individuals with CDR 0.5 and CDR 1 compared to cognitively normal CDR 0 individuals. FIGS. 2b-2d illustrate the correlation between CSF sPDGFRβ levels and albumin quotient (Qalb), CSF fibrogen, and CSF plasminogen, respectively. FIGS. 2e and 2f illustrate a representative standard curve of PDGFRβ recombinant protein measured by Western blot.

FIGS. 3a to 3g set forth the correlation of elevated baseline CSF levels of sPDGFRβ with cognitive decline in APOE4 carriers. FIG. 3a illustrates histogram frequency distribution of CSF sPDGFRβ values using median split to divide participants into two groups: high (above median) and low (below median) baseline CSF sPDGFRβ. FIGS. 3b and 3c illustrate linear mixed model analysis of study participants followed over 2-year intervals for up to 4.5 years after baseline lumbar puncture. FIGS. 3d and 3e illustrate that higher baseline CSF sPDGFRβ (dashed line) predicts future decline in mental status exam scores and global cognition after controlling for CSF Aβ and pTau status; FIGS. 3f and 3g illustrate that baseline CSF sPDGFRβ does not predict decline in either mental status (f) or global composite (g) scores in APOE3 homozygotes, regardless of CSF Aβ or pTau status.

FIGS. 4a to 4l illustrate elevated CSF sPDGFRβ, cyclophilin A and matrix metalloproteinase-9 levels in APOE4 carriers. FIG. 4a illustrates CSF sPDGFRβ levels in CDR 0 and CDR 0.5 APOE3 homozygotes and APOE4 carriers. FIG. 4b illustrates CSF sPDGFRβ levels in CDR 0 and CDR 0.5 APOE3 homozygotes and APOE4 carriers, when corrected for age, sex, education, CSF Aβ₁₋₄₂ and pTau status. FIGS. 4c and 4d illustrate the correlations between CSF sPDGFRβ and BBB Kₜᵣₐₙₛ in the hippocampus and parahippocampal gyrus. FIGS. 4e to 4g illustrate correlations between CSF sPDGFRβ and albumin quotient, fibrinogen, and plasminogen in APOE4 carriers. FIG. 4h illustrates CSF CypA in CDR 0 and CDR 0.5 bearing APOE3 and APOE4 carriers. FIG. 4i illustrates CSF cyclophilin A in CDR 0 and CDR 0.5 bearing APOE3 and APOE4 carriers, corrected for age, sex, education, CSF Aβ₁₋₄₂ and pTau status. FIG. 4j illustrates the correlation between CSF CypA and sPDGFRβ in APOE4 carriers. FIG. 4k illustrates CSF MMP9 in CDR0 and CDR 0.5 APOE3 homozygotes and APOE4 carriers. FIG. 4l illustrates the correlation between CSF MMP9 and CypA in APOE4 carriers.

### DETAILED DESCRIPTION

The detailed description of exemplary embodiments references the accompanying drawing figures, which show exemplary embodiments by way of illustration and their best mode. While these exemplary embodiments are described in sufficient detail to enable those persons skilled in the art to practice the invention, it should be understood that other embodiments may be realized and that logical, chemical, and mechanical changes may be made without departing from the spirit and scope of the inventions detailed herein. Thus, the detailed description is presented for purposes of illustration only and not of limitation. For example, unless otherwise noted, the steps recited in any of the method or process descriptions may be executed in any order and are not necessarily limited to the order presented. Furthermore, any reference to singular includes plural embodiments, and any reference to more than one component or step may include a singular embodiment or step. Also, any reference to attached, fixed, connected or the like may include permanent, removable, temporary, partial, full and/or any other possible attachment option. Additionally, any reference to without contact (or similar phrases) may also include reduced contact or minimal contact.

### Definitions

As used herein, the term “biofluid” is meant to include all physiological fluids that can be sampled from an individual. In the broadest sense, the term “biofluid” refers to CSF, blood serum, blood plasma, and urine.

As used herein, the term “platform” refers generally to an immunoassay system, generally comprising an ELISA format. The platform may comprise sandwich assays, competitive assays or antigen down assays, and may further comprise detection and measurement of absorbance, fluorescence, or chemiluminescent. In some examples, the platform may be Meso Scale Discovery (MSD), which is a multiplexed technology based on a multiple array. Various immunoassay platforms for use herein are summarized in K. L. Fox, et al., “Immunoassay Methods,” 2012 May 1 [Updated 2019 Jul. 8]. In: Sittampalam G S, Grossman A, Brimacombe K, et al., editors. Assay Guidance Manual [Internet]. Bethesda (Md.): Eli Lilly & Company and the National Center for Advancing Translational Sciences; 2004. Available online at: https://www.ncbi.nlm.nih.gov/books/NBK92434/.

As used herein, the term “immunoassay detection reagent” refers generally to any reagent capable of promoting detection of a detection antibody in an immunoassay. One or more of such reagents may be used in combination to initiate a detectable signal from a detection antibody, such as a visible light emission. In various embodiments, the molecule comprises a functional group for click chemistry at one site in the molecule and a reactive substituent at another site in the molecule that is capable of providing a light emission, such as fluorescence or chemiluminescence. In various embodiments, the functional group for conjugation to a detection antibody may comprise, but is not limited to, an azide, alkyne, nitrone, alkene, tetrazine, tetrazole or streptavidin. In various embodiments, the reactive functionality may comprise any chemical moiety capable of light emission, like fluorescence or chemiluminescence. In various embodiments, the immunoassay detection reagent comprises a sulfur-tagged molecule wherein the portion of the immunoassay detection reagent capable of light emission comprises a sulfur-containing moiety, such as a sulfonic acid, thiocyanate, sulfide, disulfide, or sulfacetamide group. In various embodiments, the immunoassay detection reagent may allow detection of biotinylated detection antibodies by conjugating to the biotinylated detection antibody and then participating in a reaction that causes a light emission. In various embodiments, the immunoassay detection reaction comprises a sulfur-tagged streptavidin wherein the sulfur tag is capable of chemiluminescence and the streptavidin is capable of conjugation to biotin. In various embodiments, sulfur-tagged streptavidin immunoassay detection reagent for use herein comprises the MSD SULFO-TAG® labeled streptavidin reagent, available from MSD, Rockville, Md., which is usable to report biotin-labeled molecules such as biotinylated detection antibodies. In various embodiments, an immunoassay detection reagent comprises a horseradish peroxidase (HRP)-conjugated streptavidin, such as available from Thermo Fisher Scientific, Waltham, Mass.

In various embodiments of the present disclosure, a new assay to detect the soluble extracellular domain of PDGFRβ using electrochemiluminescence detection on the MSD platform has been developed. To develop the assay, combinations of reagents and conditions were tested, optimized, and validated.

In various embodiments, the following reagents were used in various combinations to develop the assay: Standard bind 96-well plates (Catalog no. L15XA-3, MSD, Rockville, Md.); High bind 96-well plates (Catalog no. L15XB-1/L11XB-1, MSD); human PDGFRβ polyclonal goat IgG against amino acids Leu 33-Phe 530, and having an amino acid substitution of (Glu241Asp), (Catalog no. AF385, R&D Systems, Minneapolis, Minn.); human PDGFRβ polyclonal goat IgG biotinylated antibody against amino acids Leu 33-Phe 530, and having an amino acid substitution (Glu241Asp), (Catalog no. BAF385, R&D Systems); recombinant PDGFRβ human protein without catalytic activity domain (Catalog no. 10514H08H50, Invitrogen, Carlsbad, Calif.); carrier free recombinant human PDGFRβ Fc chimera (Catalog no. 385-PR/CF, R&D Systems); Blocker B (Catalog no. R93BB-2, MSD); SULFO-TAG® streptavidin (Catalog no. R32AD, MSD); Read Buffer T with surfactant (Catalog no. R92TC-3, MSD); adhesive seal (Microseal®, Catalog no. MSB1001, Bio-Rad, Hercules, Calif.).

### Aspects and Embodiments of the Disclosure

In various embodiments of the present disclosure, a method for determining a concentration of soluble platelet-derived growth factor R (sPDGFRβ) in a biofluid sample from a human subject is provided. The method involves forming a ternary complex of a detection antibody comprising a labelled anti-human PDGFRβ biotinylated antibody, sPDGFRβ present in the biofluid sample, and a capture antibody comprising an anti-human PDGFRβ antibody, wherein the anti-human PDGFRβ antibody is bound to a surface; detecting an intensity of light emission from the ternary complex; and interpolating the intensity of the light emission on a calibration curve to obtain the concentration of sPDGFRβ in the biofluid sample, wherein the labelled anti-human PDGFRβ biotinylated antibody comprises a conjugate between an immunoassay detection reagent and the anti-human PDGFRβ biotinylated antibody. In various embodiments, the method further involves treating the subject based on the results obtained from the above-described method.

In various embodiments, the capture antibody comprises a goat anti-human PDGFRβ polyclonal antibody. In various embodiments, In various embodiments, the detection antibody comprises a goat anti-human PDGFRβ biotinylated polyclonal antibody. In various embodiments, a non-goat species of antibody can also be used. Additionally, in various embodiments, a monoclonal antibody can be used. In various embodiments, the biofluid comprises human cerebrospinal fluid (CSF), blood serum or blood plasma. In various embodiments, the concentration of sPDGFRβ in the biofluid sample is from about 100 pg/mL to about 30,000 pg/mL. In various embodiments, the concentration of sPDGFRβ in the biofluid sample is from about 200 pg/mL to about 20,000 pg/mL, 300 pg/mL to about 15,000 pg/mL, or from about 400 pg/mL to about 10,000 pg/mL, or from about 500 pg/mL to about 9,000 pg/mL, or from about 600 pg/mL to about 8,000 pg/mL, or from about 700 pg/mL to about 7,000 pg/mL, or from about 800 pg/mL to about 6,000 pg/mL, or from about 900 pg/mL to about 5,000 pg/mL, or greater than about 1,000 pg/mL, or greater than about 1,500 pg/mL, or greater than about 2,000 pg/mL, or greater than about 3,000 pg/mL, or greater than about 4,000 pg/mL, or greater than about 5,000 pg/mL.

In various embodiments, the immunoassay detection reagent comprises a sulfur-tagged streptavidin reagent. In various embodiments, the labelled anti-human PDGFRβ biotinylated antibody further comprises a streptavidin-biotin conjugated electrochemiluminescence label. In various embodiments, other affinity moieties are used instead of the streptavidin-biotin combination.

In various aspects, the method further comprises applying a voltage to the ternary complex during the detecting step. In various embodiments, the surface comprises an electrode surface disposed in a well plate. In various aspects, the detecting step further comprises detection of an electrochemiluminescence intensity upon insertion of the well plate into an imager having electrochemiluminescence detection. In various embodiments, the calibration curve comprises an x/y plot of electrochemiluminescence intensity versus sPDGFRβ concentration.

In various embodiments, the capture antibody is bound to a bottom of the well plate by spot-coating the bottom of the well plate with a phosphate buffered solution comprising a goat anti-human PDGFRβ polyclonal antibody and polysorbate 20. In various embodiments, the ternary complex is formed in a two-step process consisting of: (a) exposing the bound goat anti-human PDGFRβ polyclonal antibody in the well plate to a diluted aliquot of the biofluid sample to form a binary complex of sPDGFRβ and the capture antibody; and (b) exposing the binary complex to a solution comprising a labelled goat anti-human PDGFRβ biotinylated polyclonal antibody.

In various embodiments, the presence of sPDGFRβ in the biofluid sample provides a pericyte injury biomarker indicative of brain microvascular and blood brain barrier (BBB) injury. In various embodiments, the presence of sPDGFRβ in the biofluid sample indicates presence of at least one neurodegenerative disorder selected from Parkinson's Disease, Huntington's Disease, Human Immunodeficiency Virus (HIV)-dementia, Post-Traumatic Brain Syndrome, or Alzheimer's disease.

In various embodiments, the immunoassay detection reagent comprises horseradish peroxidase (HRP)-conjugated streptavidin. In various embodiments, the calibration curve comprises an x/y plot of absorbance versus sPDGFRβ concentration.

In various embodiments, a method of determining the presence of cognitive impairment or dementia in a human subject is provided; the method comprising obtaining a concentration of sPDGFRβ in a biofluid sample obtained from the human subject wherein the subject is categorized as having cognitive impairment or dementia if the sPDGFRβ in the biofluid sample is greater than about 4,000 pg/mL; wherein the concentration of sPDGFRβ in the biofluid sample is obtained by: forming a ternary complex of a detection antibody comprising a labelled anti-human PDGFRβ biotinylated antibody, sPDGFRβ present in the biofluid sample, and a capture antibody comprising an anti-human PDGFRβ antibody, wherein the anti-human PDGFRβ antibody is bound to a surface; detecting an intensity of light emission from the ternary complex; and interpolating the intensity of the light emission on a calibration curve to obtain the concentration of sPDGFRβ in the biofluid sample, wherein the labelled anti-human PDGFRβ biotinylated antibody comprises a conjugate between an immunoassay detection reagent and the anti-human PDGFRβ biotinylated antibody. In various embodiments, the human subject is categorized as having dementia if the sPDGFRβ in the biofluid sample from the subject is greater than about 5,000 pg/mL. In various embodiments, the method further involves treating an individual having cognitive impairment or dementia.

In various embodiments, a method of determining the presence of Alzheimer's disease in a human subject is provided; the method comprising obtaining a concentration of sPDGFRβ in a biofluid sample obtained from the human subject, wherein the subject is categorized as having Alzheimer's disease if the sPDGFRβ in the biofluid sample is greater than about 4,000 pg/mL; wherein the concentration of sPDGFRβ in the biofluid sample is obtained by: forming a ternary complex of a detection antibody comprising a labelled anti-human PDGFRβ biotinylated antibody, sPDGFRβ present in the biofluid sample, and a capture antibody comprising an anti-human PDGFRβ antibody, wherein the anti-human PDGFRβ antibody is bound to a surface; detecting an intensity of light emission from the ternary complex; and interpolating the intensity of the light emission on a calibration curve to obtain the concentration of sPDGFRβ in the biofluid sample, wherein the labelled anti-human PDGFRβ biotinylated antibody comprises a conjugate between an immunoassay detection reagent and the anti-human PDGFRβ biotinylated antibody. In various embodiments, the human subject is categorized as having Alzheimer's disease if the sPDGFRβ in the biofluid sample from the subject is greater than about 5,000 pg/mL. In various embodiments, the method further involves treating an individual having Alzheimer's disease.

In various embodiments, an assay system for determining a concentration of soluble platelet-derived growth factor R (sPDGFRβ) in a biofluid sample is provided; the assay system comprising: a ternary complex of a detection antibody comprising a labelled goat anti-human PDGFRβ biotinylated polyclonal antibody, sPDGFRβ present in the biofluid sample, and a capture antibody comprising a goat anti-human PDGFRβ polyclonal antibody, wherein the goat anti-human PDGFRβ polyclonal antibody is bound to a surface, and wherein the labelled goat anti-human PDGFRβ biotinylated antibody is a conjugation product of an immunoassay detection reagent and the goat anti-human PDGFRβ biotinylated polyclonal antibody.

### Example 1: sPDGFRβ Assay

In various embodiments, an assay in accordance with the present disclosure comprises formation of a detectable ternary complex of sPDGFRβ analyte and antibodies. In various embodiments, the assay is a self-sandwich assay wherein both the capture and detection antibodies are the same, and are goat anti-human PDGFRβ polyclonal antibodies. In various embodiments, the biofluid sample to be analyzed for sPDGFRβ comprises CSF, blood serum or blood plasma. In various embodiments, the assay comprises the MSD platform.

First, standard-bind 96-well plates were coated with a capture antibody against the extracellular domain of human PDGFRβ. Each well was spot-coated with five μL of 40 μg/mL of human PDGFRβ polyclonal goat IgG prepared in 0.01 M phosphate-buffered saline (PBS) pH 7.4+0.03% Triton X-100. The plate was placed uncovered on a flat surface to allow the spot coating solution to air-dry overnight at room temperature. The plates were blocked with 150 μL per well of 1% Blocker B or an equivalent milk-based solution prepared in 0.01 M PBS pH 7.4+0.05% Tween-20. The plate was sealed with an adhesive seal and incubated at room temperature for 1 hour on an orbital plate shaker (˜500 rpm). The plate was washed three times with 200 μL/well of wash buffer (0.01 M PBS pH 7.4+0.05% Tween-20) and tapped on an absorbent pad to remove residual wash buffer. Blocker B diluent (0.2%) was prepared in wash buffer immediately before use and used to dilute standards and samples.

For the standard, human PDGFRβ recombinant protein without catalytic activity domain was used at a stock concentration of 0.5 μg/μL. The following standard concentrations were prepared and used in the assay: 6400, 3200, 1600, 800, 400, 200, 100 pg/mL. The diluent was used as the zero standard. Standards were mixed well by vortexing between each step. In variations of the assay, other standards may be used, such as for example, recombinant hPDGFRβ Fc Chimera Protein. For human CSF samples, 1:2 dilutions in 0.2% Blocker B diluent were prepared in polypropylene protein low-bind tubes. Twenty-five μL of prepared standards or samples were pipetted into pre-designated wells in duplicate. The plate was sealed and incubated at 4° C. overnight on an orbital plate shaker (˜500 rpm). The plate was washed three times with 200 μL/well of wash buffer and tapped on an absorbent pad to remove residual wash buffer.

The detection antibody solution was prepared by combining 1 μg/mL of human PDGFRβ biotinylated antibody, and 1 μg/mL of MSD SULFO-TAG® labeled streptavidin in 0.2% Blocker B diluent; prepared on ice immediately before use. In this example, the human PDGFRβ biotinylated antibody consisted of goat anti-human PDGFRβ polyclonal IgG. Twenty-five μL of the detection antibody solution was pipetted into each well, and the sealed plate was incubated at room temperate for 1.5 hours on an orbital plate shaker (˜500 rpm). The plate was washed three times with 200 μL/well of wash buffer and tapped on an absorbent pad to remove residual wash buffer. Read Buffer T (2×) with surfactant was prepared in ddH₂O, and 150 μL was pipetted into each well carefully avoiding the introduction of air bubbles. The plate was read immediately on the MSD SECTOR Imager 6000 equipped with electrochemiluminescence detection. The raw readings were analyzed by subtracting the average background value of the zero standard from each recombinant standard and sample readings. A standard curve was constructed by plotting the recombinant standard readings and their known concentrations and applying a linear curve fit. The sPDGFRβ concentrations in the biofluid samples were calculated using the samples' reading and the linear standard curve equation in an interpolation; the result was corrected for the sample dilution factor to arrive at the sPDGFRβ concentration in the original CSF samples. For other platforms, the detection system may be something other than the MSD Imager, and the corresponding standard curve for interpolating unknown sPDGFRβ concentrations may be, for example, an x/y plot of absorbance (at a particular wavelength or range of wavelengths) versus sPDGFRβ concentration, or fluorescent light emission versus sPDGFRβ concentration.

In a variation of the above-described assay, the sulfur-tagged immunoassay detection reagent is replaced with horseradish peroxidase (HRP)-conjugated streptavidin and 3,3′,5,5′-tetramethyl benzidine (TMB) substrate for detection of a colorimetric signal. In this variation, the MSD platform is not used at all, and the detection system instead comprises a colorimeter.

Human Study Participants

Participants were recruited through the University of Southern California (USC) Alzheimer's Disease Research Center (ADRC) in Los Angeles, Calif., and the Washington University Knight ADRC in St. Louis, Mo. A total of 147 individuals are included in this study. The study procedures were approved by the Institutional Review Boards of USC and Washington University. Participants received a lumbar puncture (LP) and venipuncture, and were evaluated using the Uniform Data Set (UDS) and additional neuropsychological tests. Participants' Clinical Dementia Rating (CDR) score was obtained through standardized interview and assessment with the participant following UDS procedures, and interview with a knowledgeable informant.

Volunteers with i) dementia (CDR>1), head injury with loss of consciousness >15 minutes, stroke, or substance abuse, or ii) current: organ failure, psychiatric or neurological disorders that might produce dementia symptoms, hydrocephalus, B12 deficiency, hypothyroidism, and medication use likely to affect brain function were excluded from the study.

Collection of Biofluids

Participants underwent lumbar puncture and venipuncture in the morning following an overnight fast. The CSF was collected in polypropylene tubes, processed (Centrifuged at 2000 g, 10 minutes, 4° C.), aliquoted into polypropylene tubes, and immediately stored at −80° C. until assay. Blood was collected into ethylenediaminetetraacetic acid (EDTA) tubes and processed (Centrifuged at 2000 g, 10 minutes, 4° C.). Plasma and the buffy coat were aliquoted in polypropylene tubes and stored at −80° C.; buffy coat was used for DNA extraction and APOE genotyping.

APOE Genotyping

DNA was extracted from buffy coat using the Quick-gDNA Blood Miniprep Kit (Catalog no. D3024, Zymo Research, Irvine, Calif.). APOE genotyping was performed via polymerase chain reaction (PCR)-based retention fragment length polymorphism analysis.

Molecular Biofluid Assays

Albumin quotient (Qalb, the ratio of CSF-to-plasma albumin levels) was determined using enzyme-linked immunosorbent assay (ELISA) (Catalog no. E-80AL, Immunology Consultants Laboratory, Inc., Portland, Oreg.). CSF levels of fibrinogen were determined by ELISA (Catalog no. E-80FIB, Immunology Consultants Laboratory, Inc.). CSF levels of plasminogen were determined by ELISA (Catalog no. E-80PMG, Immunology Consultants Laboratory, Inc.).

Statistical Analysis

For comparison between two groups, statistical significance was analyzed by unpaired two-tailed Student's t-test. For multiple comparisons, one-way analysis of variance (ANOVA) followed by Tukey's posthoc test was used. Linear regression analysis was used to assess the significance of correlations, and the Pearson correlation coefficient was determined. P<0.05 was considered significant. Statistical analyses were conducted using GraphPad Prism 7.0 software. Single data points are plotted in the figures.

Results and Discussion

Table 1 summarizes the reagents tested (i.e., plate types, block solutions, recombinant standards, capture antibodies, and detection antibodies) and identifies the combination of conditions that yielded optimal results (denoted with asterisks). Two different recombinant PDGFRβ standard proteins exhibited a large, dynamic linear curve fit ranging from 100-26,000 pg/mL with a coefficient of linearity (r²) of 0.9996 and 0.996. In Table 1, an asterisk denotes the reagent combination that yielded optimal results. Specifically, the combination of conditions that yielded optimal results are: (1) standard-bind plate type; (2) milk-based block solution; (3) recombinant standard comprising: recombinant hPDGFRβ Fc Chimera Protein, carrier free; and recombinant hPDGFRβ without catalytic activity domain; (4) capture antibody comprising: hPDGFRβ polyclonal goat IgG; and (5) detection antibody comprising: biotinylated hPDGFRβ polyclonal goat IgG and sulfur-tagged streptavidin.

TABLE 1
Summary of reagents used to develop and optimize
the sPDGFRβ assay on the MSD platform.

Plate Type
Standard-bind*
High-bind
Block Solution
Milk-based*
BSA-based
Recombinant Standard
Recombinant hPDGFRβ Fc Chimera Protein, carrier free
(R&D Systems #385-PR/CF)*
Recombinant hPDGFRβ without catalytic activity domain
(invitrogen #10514H08H50)*
Capture Antibody
hPDGFRβ monoclonal mouse IgG (Thermo #MA5-15103)
hPDGFRβ monoclonal mouse IgG (R&D Systems #MAB1263)
hPDGFRβ monoclonal mouse IgG (R&D Systems #MAB385)
hPDGFRβ polyclonal rabbit IgG (Thermo #PA1-30317)
hPDGFRβ polyclonal goat IgG (R&D Systems AF385)*
Detection Antibody
hPDGFRβ polyclonal rabbit IgG (Thermo #PA1-30317)
and Sulfo-tagged goat α rabbit IgG (MSD #R32AB)
Biotinylated mPDGFRβ polyclonal IgG (R&D Systems
#BAF1042) anti Sulfo-tagged streptavidin (MSD #R32AD)
Biotinylated hPDGFRβ polyclonal goat IgG (R&D Systems
#BAF385) and Sulfo-tagged streptavidin (MSD #R32AD)*

Two different recombinant PDGFRβ standard proteins exhibited a large, dynamic linear curve fit ranging from 100-26,000 pg/mL with a coefficient of linearity (r²) of 0.9996 and 0.996. Table 2 summarizes the parameters used to validate the performance of the PDGFRβ assay. To validate the assay, detection limits, dilutional linearity, spiked recovery, precision (including repeatability, intermediate precision, and reproducibility), and parallelism were tested.

TABLE 2
Summary of parameters used to validate performance
of the PDGFRβ assay on the MSD platform.

| Parameter | Definition | Tested |
|---|---|---|
| Detection limits | Lower and upper limits of detection are the lowest and highest | ✓ |
| | amount of analyte in a sample that can be detected, | |
| | respectively | |
| Dilution | The ability to obtain analyte concentration test results that are | ✓ |
| linearity | directly proportional to the performed dilution - validates that | |
| | sample dilution does not affect accuracy and precision | |
| Parallelism | Determines that the sample dilution response curve is parallel | ✓ |
| | to the standard concentration response curve over a range of | |
| | dilutions to ensure the test samples do not result in biased | |
| | measurements of the analyte concentration | |
| Spiked recovery | Close agreement between the accepted conventional true | ✓ |
| | analyte value (spiked) and the value found in the test sample | |
| | (recovery) | |
| Precision | Close agreement between independent test results from | ✓ |
| | replicate determinations of the same homogeneous sample | |
| | under the normal assay conditions | |
| | a. Repeatability (within-assay; within-day precision) | |
| | b. Intermediate (between-assay; between-day repeatability) | |
| | c. Reproducibility | |
| Robustness | A measure of the capacity of a method to remain unaffected | ✓ |
| | by small variations in method parameters | |

There was excellent sample recovery (average CV 2.55%) of CSF samples diluted from 1:2-1:16, indicating that the dilutions yielded consistent results within the desirable assay range. Next, parallelism measures revealed parallel response curves of samples and the standard across the dilution range, demonstrating that the test sample dilution does not result in a biased measurement of the analyte concentration.

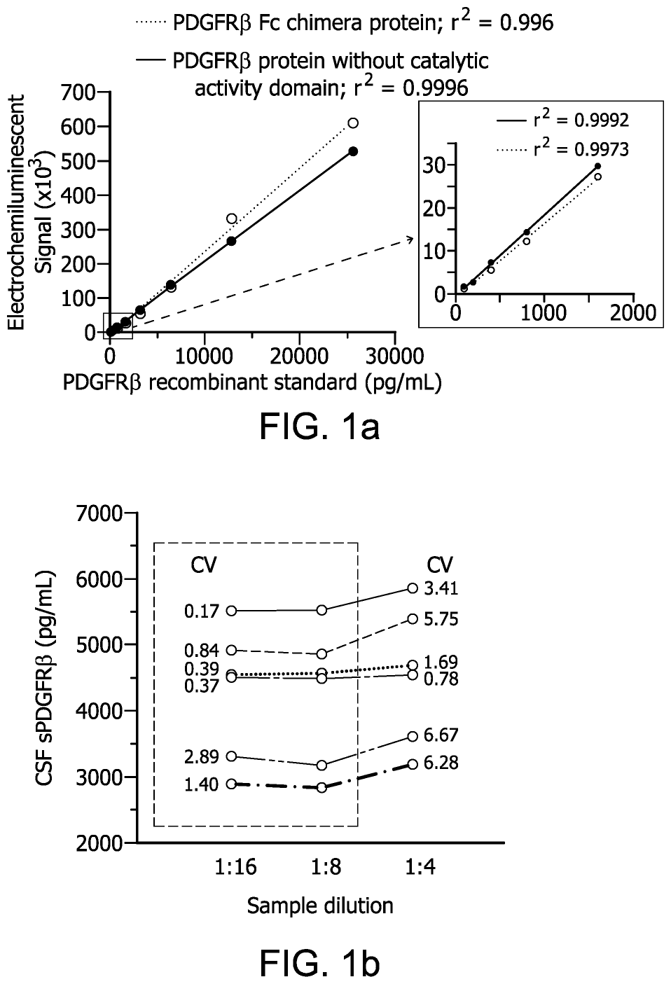

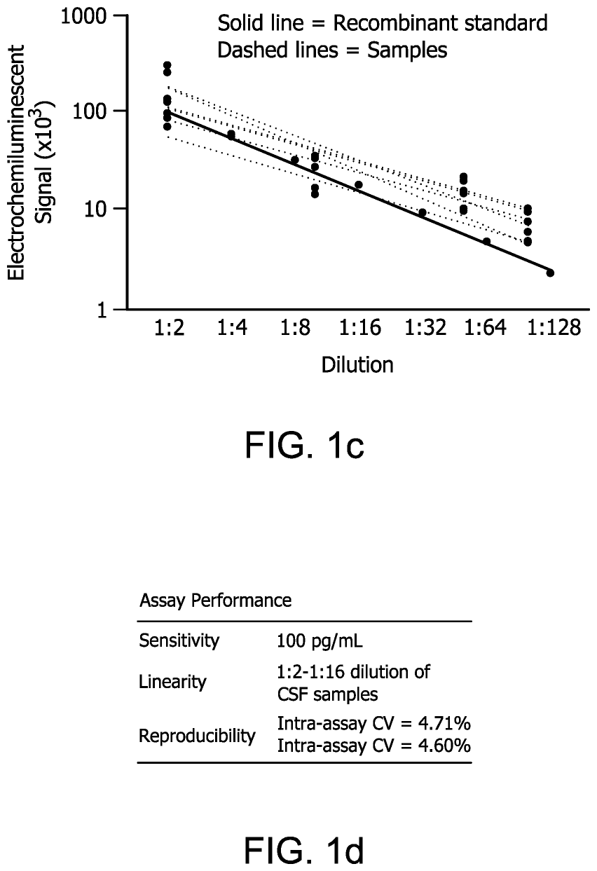

FIG. 1 sets forth the performance summary of the novel sPDGFRβ assay. In FIG. 1, FIG. 1a) sets forth representative standard curves plotting concentration and electrochemiluminescence signal of two recombinant standard proteins that both exhibit a linear curve fit over a large dynamic range from 100-26,000 pg/mL with a coefficient of linearity (r²) of 0.996-0.9996. All CSF samples measured fell within the assay's standard curve range of detection. FIG. 1b) sets forth dilution linearity test—CSF samples diluted 1:4, 1:8 and 1:16 have a low coefficient of variation (average CV 2.55%) across all sample dilutions, indicating that the dilutions yielded consistent results within the desirable assay range. FIG. 1c) shows a parallelism test—the electrochemiluminescence signal of samples and the recombinant standard protein across a range of dilutions from 1:2 to 1:128 is parallel, demonstrating that the test sample dilution does not result in a biased measurement of the analyte concentration. Precision was quantified by intra-assay and inter-assay CV of the same sample assayed under sPDGFRβ assay, resulting in an average CV of 4.71% and 4.60%, respectively. Reproducibility of the assay was tested by conducting the sPDGFRβ assay over a range of 3 years and by different laboratory personnel, which, in all instances, yielded CV<10%, which is within acceptable criteria for immunoassay CV thresholds. To validate the assay's robustness, the sPDGFRβ assay was varied by shortening the detection antibody incubation from 1.5 hours to 1 hour, and also by storing plates precoated with capture antibody for up to 1 month at 4° C. prior to conducting the assay. In both instances, the assay performance was unaffected and resulted in the same analyte concentration measured (within the <10% CV threshold) independent of the procedural variations, which indicates robustness of the assay. FIG. 1d) sets forth a summary of assay performance detailing the assay's lower limit of sensitivity (100 pg/mL), sample linearity range (1:2-1:16 dilution of CSF samples), and assay reproducibility (intra- and inter-assay variability <5%).

In summary, the new sPDGFRβ assay yields exceptional sensitivity with a lower detection limit of 100 pg/mL, and the assay produces remarkable precision and reproducibility with an average intra-assay coefficient of variability (CV) of 4.71% and an average inter-assay CV of 4.60%.

The new assay was used to evaluate sPDGFRβ levels in human CSF to test its clinical relevance. Individuals with normal cognition (CDR 0), mild cognitive impairment (CDR 0.5), and mild dementia (CDR 1) were included in the study. Table 3 presents demographic and clinical data of participants grouped by cognitive status, with the following parameters reported: CDR score, number of participants, mean age at LP, percent female, and percent APOE4 carriers.

TABLE 3
Demographic and clinical data of participants.

| | Cognitively | Cognitively | Mild Cognitive | |
|---|---|---|---|---|
| | normal, young | normai, older | Impairment | Mild Dementia |
| Clinical Dementia Rating | 0 | 0 | 0.5 | 1 |
| (CDR) scale | | | | |
| Number of participants | 14 | 59 | 36 | 38 |
| No. USC/No. WashU | 0/14 | 47/12 | 1/35 | 27/11 |
| Age at LP (mean ± SD) | 54.5 ± 6.2 | 77.45 ± 6.6 | 75.6 ± 5.9 | 76.9 ± 9.4 |
| Female, % | 50% | 59% | 36% | 50% |
| APOE4 carriers, % | 50% | 40% | 50% | 51% |

Using this assay, it was discovered that CSF sPDGFRβ levels are significantly elevated in individuals with mild cognitive impairment (CDR 0.5) and mild dementia (CDR 1) compared to cognitively normal (CDR 0) individuals, indicating brain microvascular pericyte injury during early stages of cognitive impairment. Pericyte injury and BBB breakdown are related events, as shown by positive correlations of CSF sPDGFRβ with traditional biofluid markers of BBB breakdown, including Qalb and CSF fibrinogen and plasminogen levels. Further, sPDGFRβ levels in the same CSF samples were measured by both quantitative Western blot and the new assay in accordance with the present disclosure, revealing a positive correlation as final validation of the new assay's performance.

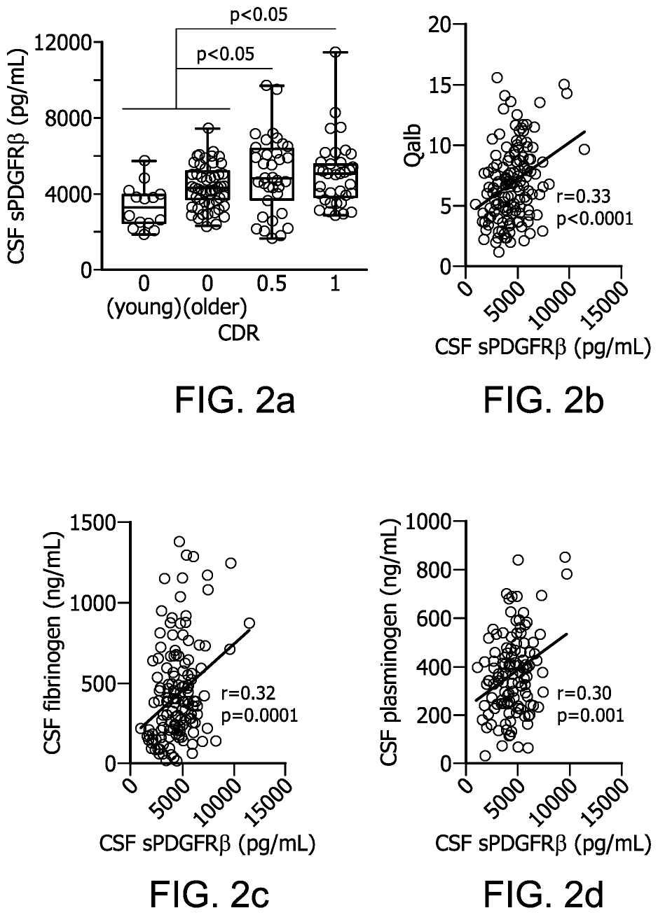

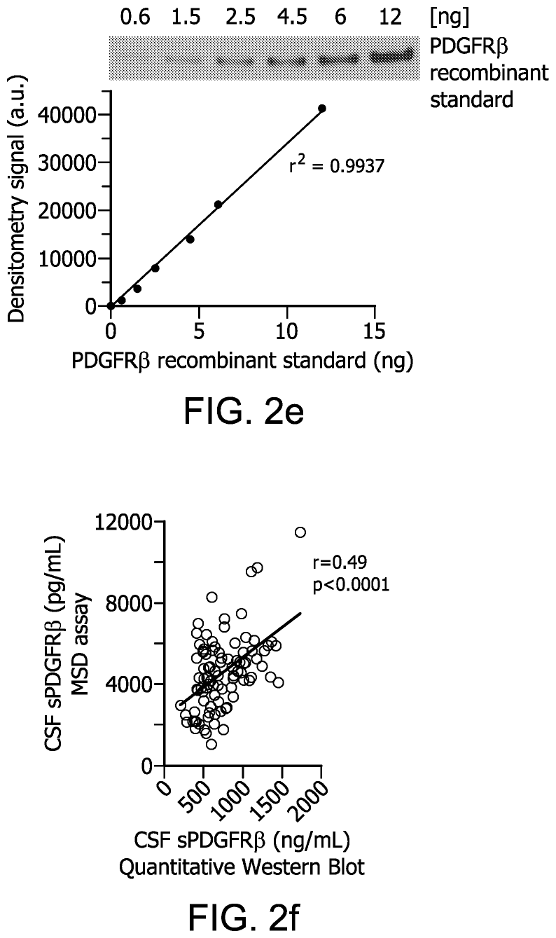

FIG. 2 sets forth the validation of sPDGFRβ as a pericyte injury biomarker in human CSF. FIG. 2a shows that CSF sPDGFRβ levels are significantly increased in individuals with CDR 0.5 (n=35) and CDR 1 (n=36) compared to cognitively normal CDR 0 individuals (n=14, young; n=59, older); significance by ANOVA with Tukey posthoc test, α=0.05. FIGS. 2b-2d indicate CSF sPDGFRβ relates to blood-brain barrier breakdown as evidenced by positive correlations with albumin quotient (Qalb) of CSF-to-plasma albumin levels (n=143)(FIG. 2b); CSF fibrinogen (n=144) (FIG. 2c); and CSF plasminogen (n=121) (FIG. 2d). FIGS. 2e and 2f show a representative standard curve of PDGFRβ recombinant protein measured by Western blot (FIG. 2e) used to quantify sPDGFRβ levels in CSF samples by quantitative Western blot in panel (FIG. 2f). There is a positive correlation of CSF sPDGFRβ levels measured by quantitative Western blot and the new assay (n=93) (FIG. 2f).

All panels plot single data points. In panel a, the box and whisker plots indicate the median value (horizontal line), the boxes indicate the interquartile range, and the whiskers indicate the minimum and maximum values. In panels b-d and f, Pearson correlation coefficient, r; significance by linear regression analysis.

The novel assay in accordance with the present disclosure is the first to offer a reproducible approach to quantify sPDGFRβ in human CSF, and these results provide important support that CSF sPDGFRβ is a promising and sensitive biomarker for identifying individuals that are at increased risk of developing early cognitive impairment. Compared with methods to detect CSF sPDGFRβ by quantitative Western blot, the new assay has a larger range of sensitivity, and more high-throughput, making it easy to incorporate at different sites to investigate pericyte injury in various cohorts.

PDGFRβ is predominantly expressed by pericytes in the adult brain of humans and mice, and sPDGFRβ is primarily shed by pericytes. Thus, increased CSF sPDGFRβ levels reflect brain microvascular damage mainly due to pericyte injury. The new assay disclosed herein detects the soluble extracellular portion of PDGFRβ, which has 5 immunoglobulin (Ig)-like domains. Ligands predominantly bind to Ig-like domains 2 and 3 causing receptor dimerization, and the receptor dimer is further stabilized by direct receptor-receptor interactions of Ig-like domain 4. To date the 3-dimensional structure of PDGFRβ has not been resolved, nor have the precise mechanism(s) of PDGFRβ ectodomain shedding from pericytes been elucidated. Recent evidence indicates that a disintegrin and metalloproteinase (ADAM) family member, ADAM10, can mediate sPDGFRβ shedding from pericytes but not SMCs, consistent studies showing ADAM10 sheds sPDGFRβ in fibroblasts. While ADAM10 plays a role in PDGFRβ shedding from pericytes, it is currently elusive whether ADAM17 or other enzymes are also involved. Further, it is presently unknown whether the extracellular domain of PDGFRβ is internalized or cleaved into the soluble form prior to receptor internalization. Elucidating the exact mechanism(s) underlying ectodomain shedding of PDGFRβ in response to pericyte injury would not only inform the degree to which sPDGFRβ is detectable as a result of pericyte dysfunction versus degeneration but also has the potential to identify novel therapeutic targets.

In light of the growing evidence that cerebrovascular dysfunction contributes to cognitive impairment and dementia, including AD, different clinical sites may adopt and employ this assay to evaluate sPDGFRβ in their cohorts of individuals with neurodegenerative disorders associated with neurovascular dysfunction and VCID. Since, sPDGFRβ is a biomarker of brain pericyte and BBB injury, this new assay will allow future diagnostic and therapeutic studies of brain microvascular damage in relation to cognition in different neurodegenerative disorders associated with neurovascular dysfunction and VCID.

In conclusion, a combination of antibodies and standards yielding a highly sensitive and reproducible sPDGFRβ assay with inter- and intra-assay coefficient of variation <5% was identified. Using this assay, significantly elevated CSF sPDGFRβ has been confirmed in individuals with mild cognitive impairment compared to cognitively normal individuals. This new assay reliably quantifies sPDGFRβ levels in human biofluids and could be easily applied at different clinical sites. The assay in accordance with the present disclosure will allow future diagnostic and therapeutic studies of brain pericyte, BBB and microvascular damage in relation to cognition in different neurological and neurodegenerative disorders associated with neurovascular dysfunction.

A summary of the advantages of the MSD-based sPDGFRβ assay over other existing approaches is summarized in Table 4. Compared with existing approaches to detect sPDGFRβ by either quantitative western blot or the only commercially available ELISA assay (Thermo Fisher Scientific), the MSD assay presented herein has favorable features such as (1) high throughput, (2) requires significantly less CSF sample volume, (3) has a large dynamic range of detection, (4) is time and cost effective, (5) has high precision and accuracy, and (6) has the capability to be multiplexed with other key analytes for research or clinical utility (Table 4). Additionally, the MSD-based sPDGFRβ assay is easy to incorporate at different laboratories to investigate the pericyte and BBB injury in various cohorts.

TABLE 4
Comparative performance of the sPDGFRβ assay
on the MSD platform versus other existing approaches.

| | Approach to measure human sPDGFRβ | | |
|---|---|---|---|
| | | Thermo | |
| | | Fisher | Novel assay |
| | Quantitative | Scientific | on MSD |
| | Western blot | ELISA | platform |
| High-throughput | No | Yes | Yes |
| CSF volume required | Moderate (25 μl) | High (100 μl) | Low (7 μl) |
| Large dynamic range | No | No | Yes |
| of detection | | | |
| Time & Cost | High | Low | Low |
| Precision & Accuracy | Moderate | High | High |
| Multiplex capability | No | No | Yes |
| Prognostic value | Low | Moderate | High |

Note:
Bold text indicates optimal performance features.

### Example 2: Correlation Between Elevated Baseline CSF Levels and Cognitive Decline

In humans with Alzheimer's disease (AD) and animal models, elevated levels of sPDGFRβ in the CSF indicate that pericyte injury is linked to BBB breakdown and cognitive dysfunction.

Study Participants

Participants were recruited from three sites: the University of Southern California (USC), Los Angeles, Calif.; Washington University (WashU), St. Louis, Mo.; and Banner Alzheimer's Institute Phoenix, Ariz. and Mayo Clinic Arizona, Scottsdale, Ariz. as a single site. At the USC site, participants were recruited through the USC Alzheimer's Disease Research Center (ADRC): combined USC and the Huntington Medical Research Institutes (HMRI), Pasadena, Calif. At the WashU site, participants were recruited through the Washington University Knight ADRC. At Banner Alzheimer's Institute and Mayo Clinic Arizona site, participants were recruited through the Arizona Apolipoprotein E (APOE) cohort. The study and procedures were approved by the Institutional Review Boards of USC ADRC, Washington University Knight ADRC, and Banner Good Samaritan Medical Center and Mayo Clinic Scottsdale, indicating compliance with all ethical regulations. Informed consent was obtained from all participants before study enrolment. All participants (n=435) underwent neurological and neuropsychological evaluations performed using the Uniform Data Set (UDS) (Morris et al. Alzheimer Dis Assoc Discord 20, 210-216 (2016)) and additional neuropsychological tests, as described below, and received a venipuncture for collection of blood for biomarker studies. An LP was performed in 350 participants (81%) for collection of CSF. DCE-MRI for assessment of BBB permeability was performed in 245 participants (56%) who had no contraindications for contrast injection. Both LP and DCE-MRI were conducted in 172 participants. Among the 245 DCE-MRI participants, 74 and 96 were additionally studied for brain uptake of amyloid and tau PET radiotracers, respectively, as described below. No statistical methods were used to predetermine sample size. All biomarker assays, MRI, and PET scans were analyzed by investigators blinded to the clinical status of the participants.

Participant Inclusion and Exclusion Criteria

Included participants (>45 years of age) were confirmed by clinical and cognitive assessments to be either cognitively normal or at the earliest symptomatic stage of AD. A current or prior history of any neurological or psychiatric conditions that might confound cognitive assessment, including organ failure, brain tumours, epilepsy, hydrocephalus, schizophrenia, and major depression, was exclusionary. Participants were stratified by APOE genotype as APOE4 carriers (ε3/ε4 and ε4/ε4) or APOE4 non-carriers (ε3/ε3), also defined as APOE3 homozygotes, who were cognitively normal or had mild cognitive dysfunction, as determined by CDR scores (Morris Neurology 43, 2412-2414 (1993)) and the presence of cognitive impairment in one or more cognitive domains based on comprehensive neuropsychological evaluation, including performance on ten neuropsychological tests assessing memory, attention/executive function, language and global cognition. For all analyses individuals with ε3/ε4 and ε4/ε4 alleles were pooled together in a single APOE4 group, as a significant difference between individuals with two versus one 84 allele for the studied parameters, including the BBB Ktrans and sPDGFRβ CSF values (see statistical section below), were not found in the present cohort (82-86% ε3/ε4 and 14-18% ε4/ε4 participants, depending on the outcome measure). Individuals were additionally stratified by Aβ and pTau CSF analysis as either Aβ1-42+(<190 pg/ml) or Aβ1-42− (>190 pg/ml), and pTau+(>78 pg/ml) or pTau− (<78 pg/ml), using accepted cutoff values (Nation et al. Nat Med 25, 270-276 (2019); Pan et al. J Alzheimers Dis 45, 709-719 (2015); Roe et al. Neurology 80, 1784-1791 (2013)).

Participants were excluded if they were diagnosed with vascular cognitive impairment or vascular dementia. Clinical diagnoses were made by neurologists and criteria included whether the patient had a known vascular brain injury, and whether the clinician judged that the vascular brain injury played a role in their cognitive impairment, and/or pattern and course of symptoms. In addition to clinical diagnosis, the presence of vascular lesions was confirmed by moderate-to-severe white matter changes and lacunar infarcts by fluid-attenuated inversion recovery. (FLAIR) MRI and/or subcortical microbleeds by T2*-weighted MRI1.

Participants were also excluded if they were diagnosed with Parkinson's disease, Lewy body dementia or frontotemporal dementia. History of a single stroke or transient ischaemic attack was not an exclusion unless it was related to symptomatic onset of cognitive impairment. Participants also did not have current contraindications to MRI and were not currently using medications that might better account for any observed cognitive impairment.

Clinical Exam

Participants underwent clinical assessments according to UDS procedures harmonized across all study sites, including clinical interview and review of any neurocognitive symptoms and health history with the participant and a knowledgeable informant. A general physical and neurologic exam was conducted. The CDR assessment was conducted in accordance with published standardization procedures, including standardized interview and assessment with the participant and a knowledgeable informant. In accordance with current diagnostic models for cognitive and biological research criteria for cognitive impairment and AD (Jack et al. Alzheimers Dement 14, 535-562 (2018)), participants were separately stratified by cognitive impairment and AD biomarker abnormality using established cutoffs for CSF Aβ1-42 and pTau (Nation et al. Nat Med 25, 270-276 (2019); Pan et al. J Alzheimers Dis 45, 709-719 (2015); Roe et al. Neurology 80, 1784-1791 (2013)). Cognitive impairment was determined on the basis of global CDR score and neuropsychological impairment in one or more cognitive domains.

Vascular Risk Factors

The vascular risk factor (VRF) burden in each participant was evaluated through physical examination, blood tests, and clinical interviews with the participant and informant; history of cardiovascular disease (heart failure, angina, stent placement, coronary artery bypass graft, intermittent claudication), hypertension, hyperlipidaemia, type 2 diabetes, atrial fibrillation, and transient ischaemic attack or minor stroke were investigated. The total VRF burden was defined by the sum of these risk factors, as previously described (Nation et al. Nat Med 25, 270-276 (2019)). An elevated VRF burden was assigned to individuals with two or more VRFs. This threshold was adopted because previous studies showed that the presence of two or more VRFs is associated with occult cerebrovascular disease at autopsy in older adults with AD, whereas a single VRF is common and not necessarily associated with increased cerebrovascular disease in this population.

Cognitive Domain Impairment Evaluation

Impairment in one or more cognitive domain was judged by performance on comprehensive neuropsychological testing, using previously described neuropsychological criteria for cognitive impairment described (Nation et al. Nat Med 25, 270-276 (2019)). All participants underwent neuropsychological testing that included the UDS battery (version 2.0 or 3.0) plus supplementary neuropsychological tests at each site. Raw test scores were converted to age-, sex- and education-corrected z scores using the National Alzheimer's Coordinating Center (NACC) regression-based norming procedures (https://www.alz.washington.edu/). Normalized z scores from ten neuropsychological tests were evaluated in determining domain impairment, including three tests per cognitive domain (memory, attention/executive function and language) and one test of global cognition. Impairment in one or more cognitive domains was determined using previously described neuropsychological criteria, and was defined as a score >1s.d. below norm-referenced values on two or more tests within a single cognitive domain or three or more tests across cognitive domains (Jak et al. Am J Geriatr Psychiatry 17, 368-375 (2009)). Prior studies have established improved sensitivity and specificity of these criteria relative to those employing a single test score, as well as adaptability of this diagnostic approach to various neuropsychological batteries (Jak et al. Am J Geriatr Psychiatry 17, 368-375 (2009); Jak et al. J Int Neuropsychol Soc 22, 937-943 (2016)). Participants were excluded from cognitive domain analyses if they had less than 90% complete neuropsychological test data (53, 24, and 82 participants were excluded for MRI, PET, and CSF analyses, respectively). Included participants were classified as 0, 1, or 2+ based on the number of cognitive domains for which they had two or more impaired test scores.

Test battery specifics for each UDS version and recruitment site are as follows. i) Global cognition: MMSE for UDS version 2 (Weintraub Alzheimer Dis Assoc Disord 23, 91-101 (2009)) and MoCA for UDS version 3 (Besser et al. Alzhiemer Dis Assoc Disord 32, 351-358 (2018)). ii) Memory: The Logical Memory Story A Immediate and Delayed free recall tests (modified from the original Wechsler Memory Scales, Third Edition (WMS-III)) for UDS version 2 and the Craft Stories Immediate and Delayed free recall for UDS version 3. For supplementary tests the USC participants underwent the California Verbal Learning Test, Second Edition (CVLT-II) and the Selective Reminding Test (SRT) sum of free recall trials. Norm-referenced scores for these supplementary test scores were derived from a nationally representative sample published with the test manual (CVLT-II) (delis et al. California Verbal Learning Test (PsychCorp, 2000)) and in studies of normally ageing adults (SRT). iii) Attention and executive function: The Trails A, Trails B, and Wechsler Adult Intelligence Scale-Revised (WAIS-R) Digit Span Backwards tests for UDS version 2 and the Trails A, Trails B and Digit Span Backwards tests for UDS version 3. iv) Language: The Animal Fluency, Vegetable Fluency, and Boston Naming Tests for UDS version 2 and Animal Fluency, Vegetable Fluency, and Multilingual Naming Test (MINT) for UDS version 3.

Lumbar Puncture and Venipuncture

Participants underwent a lumbar puncture and venipuncture in the morning after an overnight fast. The CSF was collected in polypropylene tubes, processed (centrifuged at 2,000 g, 4° C., 10 min USC site; 5 min WashU site), aliquoted into polypropylene tubes and stored at −80° C. until assay. Blood was collected into EDTA tubes and processed (centrifuged at 2,000 g, 4° C., 10 min USC site; 5 min WashU site). Plasma and buffy coat were aliquoted in polypropylene tubes and stored at −80° C.; buffy coat was used for DNA extraction and APOE genotyping.

APOE Genotyping

DNA was extracted from buffy coat using the Quick-gDNA Blood Miniprep Kit (catalogue no. D3024, Zymo Research, Irvine, Calif.). APOE genotyping was performed via polymerase chain reaction (PCR)-based retention fragment length polymorphism analysis, as previously reported (Nation et al. Nat Med 25, 270-276 (2019)).

Molecular Assays

Quantitative western blotting of sPDGFRβ. The quantitative western blot analysis was used to detect sPDGFRβ in human CSF (ng/ml), as previously reported (Nation et al. Nat Med 25, 270-276 (2019); Montagne et al. Neuron 85, 295-302 (2015)).

BBB breakdown biomarkers. Albumin quotient (Qalb, the ratio of CSF to plasma albumin levels) and CSF levels of fibrinogen and plasminogen were determined using enzyme-linked immunosorbent assay (ELISA), as previously reported (Nation et al. Nat Med 25, 270-276 (2019); Montagne et al. Neuron 85, 295-302 (2015)).

Cyclophilin A. A CypA assay was developed on the Meso Scale Discovery (MSD) platform. Standard-bind 96-well plates (catalogue no. L15XA-3/L11XA-3, MSD, Rockville, Md.) were spot-coated with 5 μl per well of 40 μg/ml rabbit polyclonal anti-CypA antibody (catalogue no. 10436-T52, Sino Biological, Wayne, Pa.) prepared in 0.03% Triton X-100 in 0.01 M PBS pH 7.4 solution. The plates were left undisturbed overnight to dry at room temperature. The next day, the plates were blocked with 150 μl per well of Blocking One (catalogue no. 03953-95, Nacalai Tesque, Japan) and incubated for exactly 1 h with shaking. Meanwhile, samples and standards were prepared in Blocking One blocking buffer. Different concentrations ranging from 3.5 to 200 ng/ml of a recombinant human CypA protein (catalogue no. 3589-CAB, R&D Systems, Minneapolis, Minn.) were used to generate a standard curve. All CSF samples were diluted 1:3. After blocking, the plates were manually washed three times with 200 μl per well of wash buffer (in 0.05% Tween-20 in 0.01 M PBS pH 7.4). The prepared samples or standards were added at 25 μl per well, and the plates were incubated overnight at 4° C. with shaking.

The next day, the plates were washed three times, and 25 μl per well of 1 μg/ml sulfo-tagged mouse monoclonal CypA detection antibody (catalogue no. ab58144, Abcam, Cambridge, Mass.), prepared in Blocking One. The plates were incubated for 90 min at room temperature with shaking. Next, the plates were washed four times, then 150 μl per well of 2×Read Buffer T with surfactant (catalogue no. R92TC-3, MSD, Rockville, Md.) was added and the plates were read immediately on an MSD SECTOR Imager 6000 (MSD, Rockville, Md.) with electrochemiluminescence detection.

The raw readings were analysed by subtracting the average background value of the zero standard from each recombinant standard and sample reading. A standard curve was constructed by plotting the recombinant standard readings and their known concentrations and applying a nonlinear four-parameter logistics curve fit. The CypA concentrations were calculated using the samples' reading and the standard curve equation; the result was corrected for the sample dilution factor to arrive at the CypA concentration in the CSF samples.

Matrix metalloproteinase-9. CSF levels of MMP9 were determined using the human MMP9 Ultra-Sensitive Kit from MSD (cat. No. K151HAC). Neuron-specific enolase. CSF levels of NSE were determined using ELISA (cat. no. E-80NEN, Immunology Consultant Laboratories, Portland, Oreg.). The company no longer sells this product; thus, this analyte was measured in the majority of participants but not in those individuals that enrolled in the study most recently.

S100B. CSF levels of the astrocyte-derived cytokine, S100 calcium-binding protein B (S100B), were determined using ELISA (cat. no. EZHS100B-33K, EMD Millipore, Billerica, Mass.).

Inflammatory markers. An MSD multiplex assay was used to determine CSF levels of intercellular adhesion molecule 1 (ICAM1) (cat. no. K15198D, MSD, Rockville, Md.), and interleukin-6 (IL6), IL-1β, tumour necrosis factor-α (TNFα), and interferon gamma (IFNγ) (cat. no. K15049G, MSD, Rockville, Md.).

Aβ peptides. An MSD multiplex assay (cat. no. K15200E, MSD, Rockville, Md.) was used to determine CSF levels of Aβ₁₋₄₂. Participants were stratified based on CSF analysis as either Aβ+(<190 pg/ml) or Aβ−(>190 pg/ml) using the accepted cutoff values as previously reported for the MSD 6E10 Aβ peptide assay (Pan et al. J Alzheimers Dis 45, 709-719 (2015)).

Tau. Phosphorylated tau (pT181) was determined by ELISA (cat. no. 81581, Innotest, Fujirebio US, Inc., Malvern, Pa.). Participants were stratified based on CSF analysis as either pTau+(>78 pg/ml) or pTau− (<78 pg/ml), using the accepted cutoff value as previously reported (Roe, et al. Neurology 80, 1784-1791 (2013)).

Statistical Analyses

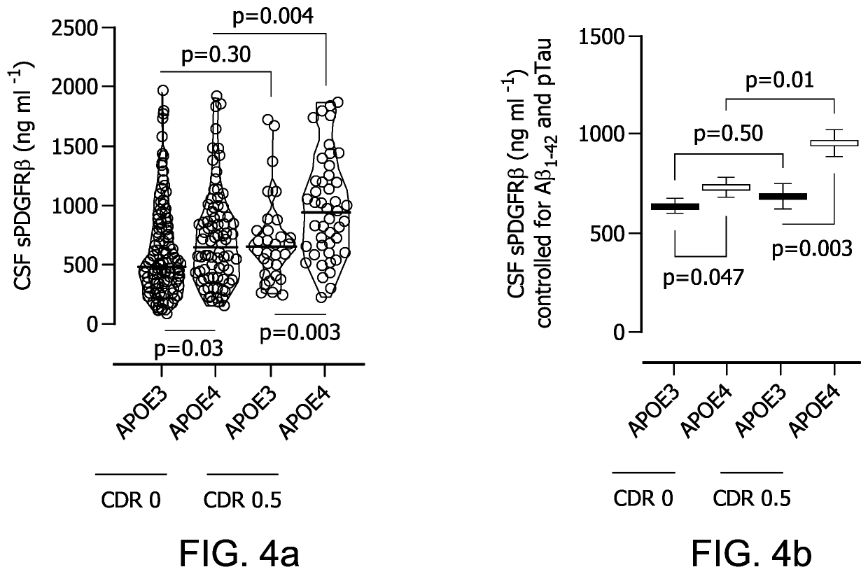

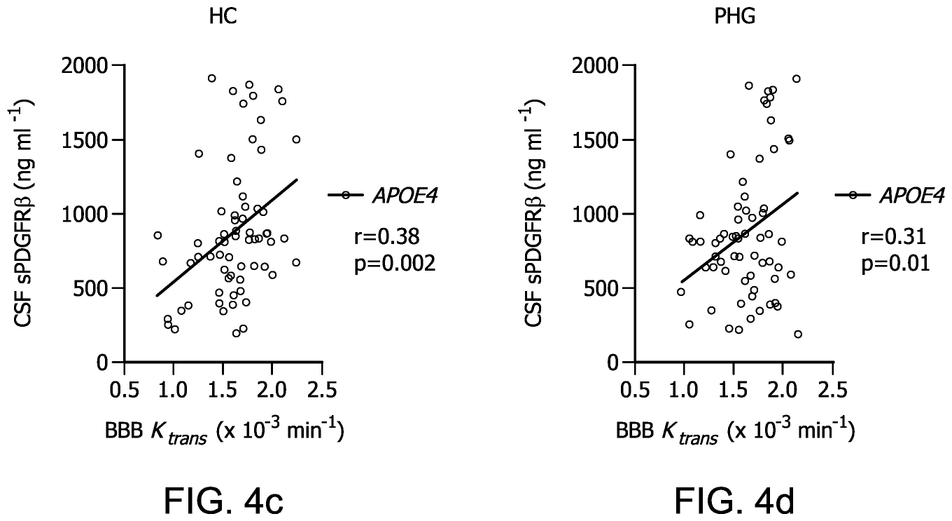

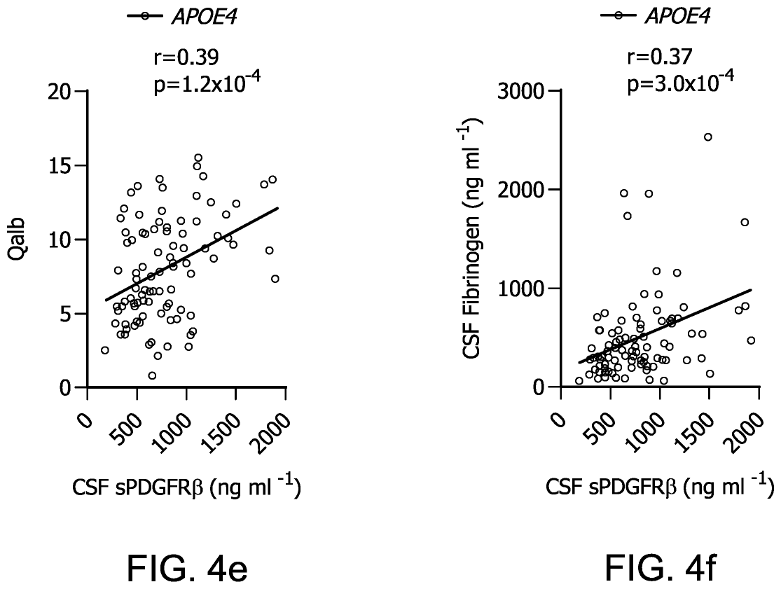

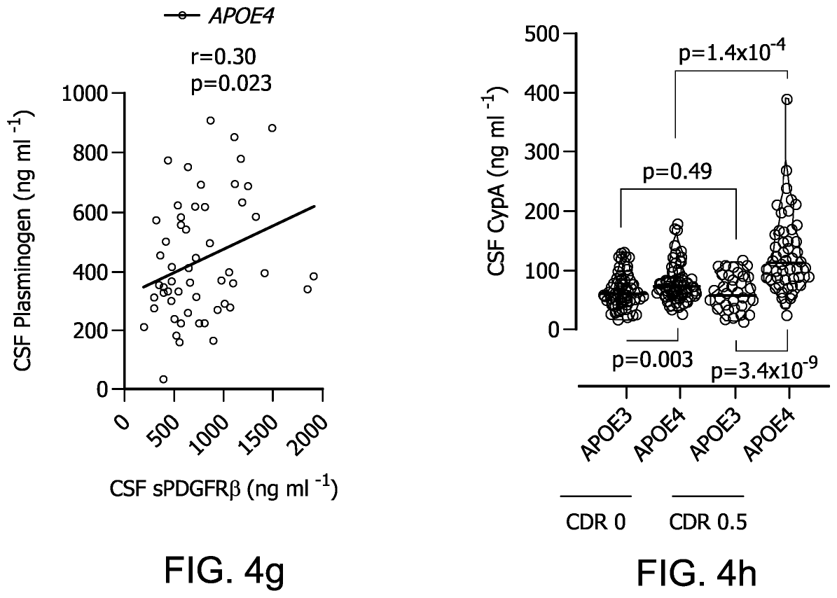

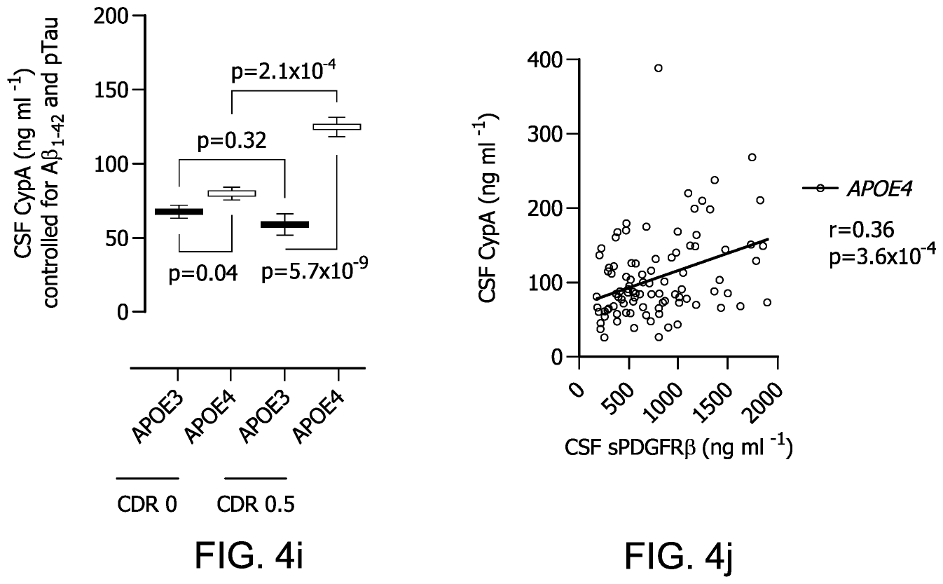

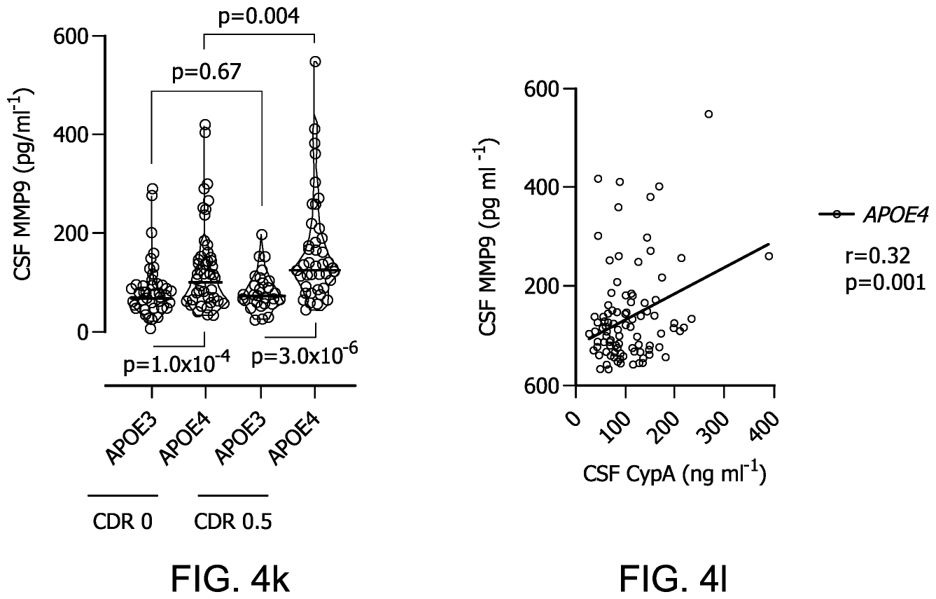

Prior to performing statistical analyses, we first screened for outliers using the Grubbs' test, also called the ESD (extreme studentized deviate) method, applying a significance level of α=0.01 (https://www.graphpad.com/quickcalcs/grubbs1/). For each of the outliers identified, a secondary index of outlier influence was applied using the degree of deviation from the mean (greater than ±3 s.d.) (Aggarwal, C. C. Outlier Analysis (Springer, 2013)). Continuous variables were also evaluated for departures from normality through quantitative examination of skewness and kurtosis, in addition to visual inspection of frequency distributions. Where departures of normality were identified, log₁₀ transformations were applied, and distribution normalization was confirmed before parametric analyses. This was done for FIGS. 4h and 4k. As the use of log₁₀ transformations accounts for any non-normality, this obviated the need for outliers exclusion.

DCE-MRI Ktrans, and CSF sPDGFRβ and CypA.

Regional DCE-MRI Kₜᵣₐₙₛ values and CSF sPDGFRβ, CypA and MMP9 levels were compared across the entire sample stratified by APOE status. As in the APOE4 group relatively few participants were homozygous ε4/ε4 compared to heterozygous ε3/ε4 (14% for DCE-MRI analysis, and 18% for sPDGFRβ analysis), and initial comparisons between ε4/ε4 and ε3/ε4 carriers did not show any significant differences in regional HC and PHG DCE-MRI Ktrans values (CDR 0, PHC=0.19 and P_{PHG}=0.54 (PHG); CDR 0.5, P_{HC}=0.22 and P_{PHG}=0.84) or CSF sPDGFRβ levels (CDR 0, P=0.23; CDR 0.5, P=0.47), all subsequent analyses combined APOE4 carriers (ε3/ε4 and ε4/ε4), and compared these participants to APOE3 carriers (ε3/ε3) stratified by cognitive impairment status (CDR 0 versus 0.5 and 0 versus 1 versus 2+ cognitive domain impairment using ANCOVA with FDR correction for multiple comparisons (see details below). For CDR analyses, model covariates included age, sex, and education. Cognitive domain impairment was determined using age-, sex-, and education-corrected values, so these covariates were not additionally included in the analyses. Additional post hoc ANCOVA analyses evaluated whether the observed differences remained significant after stratifying APOE4 carriers by CSF Aβ₁₋₄₂ and pTau status, and after statistically controlling for CSF Aβ₁₋₄₂ and pTau status and regional brain volume in APOE4 non-carriers and carriers. These findings were also confirmed by hierarchical logistic regression models using the same covariates.

Pet Ad Biomarkers.

In a subset of participants who underwent amyloid and tau PET imaging together with DCE-MRI studies, we used ANCOVA models controlled for age, sex and education to compare regional amyloid and tau ligand binding and DCE-MRI values in a set of APOE4 non-carriers and carriers within a priori regions of interest, based on prior imaging studies, to determine whether distinct regional pathologies differed by APOE4 carrier status.

Baseline CSF sPDGFRβ as a Continuous Predictor of Cognitive Decline.

For linear mixed model analysis, baseline CSF sPDGFRβ was a continuous predictor of demographically corrected global cognitive change at 2-year follow up intervals, controlling for CSF Aβ₁₋₄₂ and CSF pTau status. Global cognition was indexed by age-, sex-, and education-corrected z scores on mental status exam (MMSE or MoCA) and as the global cognitive composite of all age-, sex-, and education-corrected neuropsychological test z scores (see above for list of neuropsychological tests). Time was modelled with date of LP as baseline (t0) with two follow-up intervals of 2 years each (t1, t2). Additional analyses confirmed all findings when time was modelled as time since baseline, with date of lumbar puncture as baseline (t0) and follow up as annual intervals (t1-n).

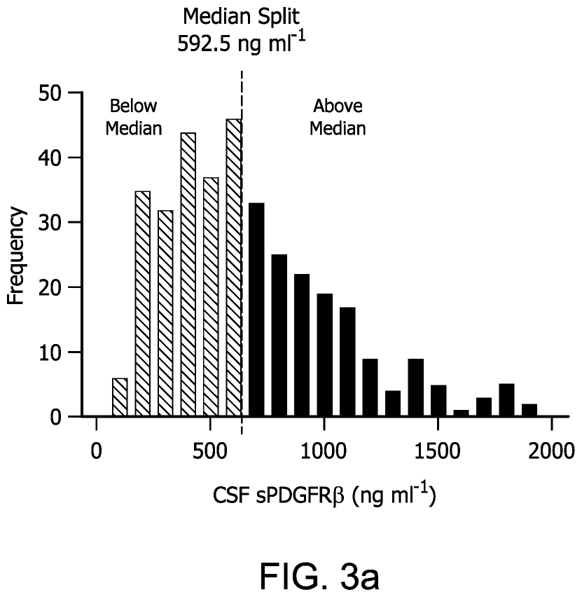

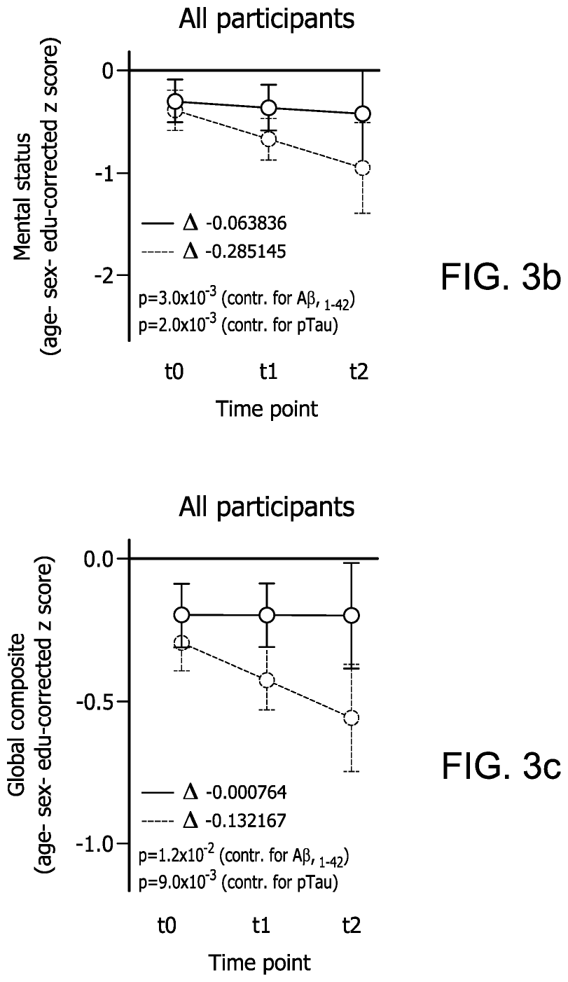

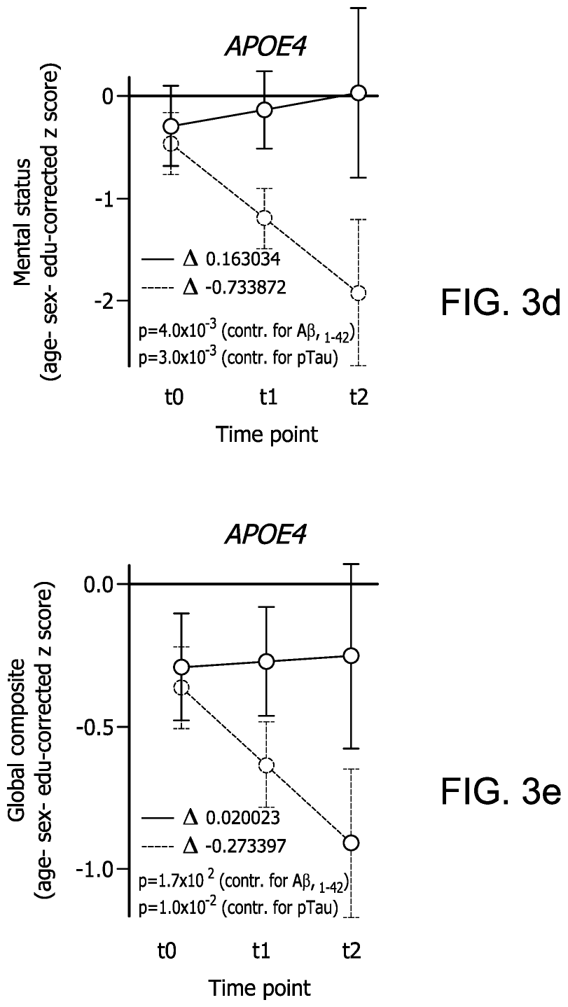

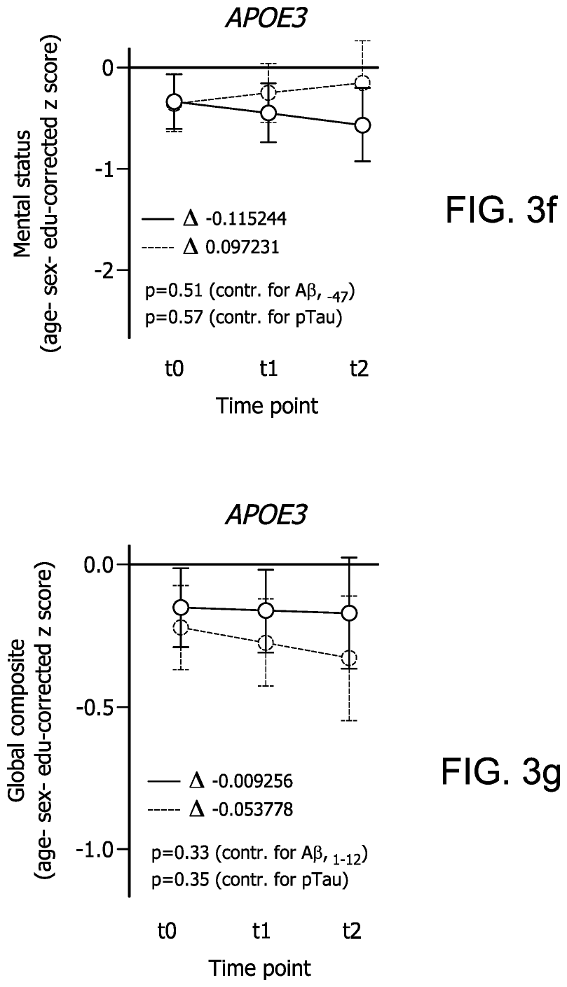

All longitudinal mixed models treated CSF sPDGFRβ as a continuous predictor. Although we have previously established that CSF sPDGFRβ is a marker of pericyte injury, the optimal cutoff value for abnormal CSF sPDGFRβ levels indicative of pericyte injury remains unknown. Autopsy studies are required to determine optimal in vivo biomarker cutoff values that predict gold-standard neuropathological measures, such as studies conducted for CSF and PET markers of amyloid and tau. Given the lack of available autopsy data relating CSF sPDGFRβ to neuropathological markers of pericyte injury, we chose to divide participants by CSF sPDGFRβ values using median split for the purposes of visual display only (higher CSF sPDGFRβ was above sample median and lower CSF sPDGFRβ was below sample median). The median split was not used in statistical analyses and was only used for the purpose of visual display (FIG. 3a) for statistical parameters from analyses using CSF sPDGFRβ as a continuous predictor of cognitive decline).

Correlational Analyses.

Pearson product moment correlations were used to evaluate relationships among CSF sPDGFRβ, CypA, MMP9, fibrinogen, plasminogen and hippocampal and parahippocampal BBB Kₜᵣₐₙₛ levels among APOE4 carriers.

Multiple Comparison Correction and Missing Data.

Given the large number of analyses, FDR correction was applied to P values for primary study outcomes (DCE-MRI, sPDGFRβ) evaluated in the entire sample by APOE4 carrier status and CDR status using the Benjamini-Hochberg method (Glickman et al. J Clin Epidemiol 67, 850-857 (2014)) in ANCOVA and logistic regression models controlling for age, sex, education, brain volume, and CSF Aβ₁₋₄₂ and pTau status (for DCE-MRI analyses). Post hoc confirmatory analyses in participant subsets further evaluating independence of CSF and PET markers of amyloid and tau, evaluation of mechanistic markers (that is, CypA and MMP9), and longitudinal analysis of predictive value of CSF sPDGFRβ were not corrected for multiple comparisons. For longitudinal data with variable follow up, we used linear mixed model analyses with and accounted for missing data via the missing at random assumption.

Results

Using a median split for visual display of the CSF sPDGFRβ baseline levels from 350 participants, all participants were stratified into two groups: low CSF sPDGFRβ levels (0-600 ng ml⁻¹) and high sPDGFRβ levels (600-2,000 ng ml⁻¹), as shown in FIG. 3a. FIG. 3a illustrates histogram frequency distribution of CSF sPDGFRβ values using median split to divide participants into two groups: high (above median 600-2,000 ng ml-1) and low (below median; 0-600 ng ml-1) baseline CSF sPDGFRβ. All longitudinal analyses used baseline CSF sPDGFRβ as a continuous predictor of future cognitive decline. In 146 APOE4 carriers and APOE3 homozygotes who were evaluated by cognitive exams at 2-year intervals up to 4.5 years from baseline lumbar puncture (LP), participants with higher baseline CSF sPDGFRβ exhibited accelerated cognitive decline on a global mental status exam and global cognitive composite z-scores, which remained significant after controlling for CSF Aβ and tau status, as shown in FIG. 3b, 3c, and Table 5. Higher baseline CSF sPDGFRβ (dashed line) predicts greater decline in demographically-corrected mental status exam scores over time (p=0.01) (this remains significant after controlling for CSF Aβ (p=0.002) and pTau (p=0.002) status; (b), and in global cognitive composite scores (p=0.01) (this remains significant after controlling for CSF Aβ (p=0.017) and pTau (p=0.01) status; (c).

TABLE 5
Linear mixed model analysis of CSF sPDGFRβ baseline values
predicting future cognitive decline on age-, sex-, and education-
corrected z-scores on mental status exam and the global cognitive
composite of all neuropsychological tests after controlling
for CSF Aβ and tau status. Significance by linear mixed
model analysis; no multiple comparison correction applied.
All tests are two-tailed. Total sample (n = 146).

| | | | | |
|---|---|---|---|---|
| β | SE | df | t | p-value |

CSF sPDGFRβ Predicting Change in Mental Status
Controlling for CSF Aβ₁₋₄₂ and pTau

| | | | | | |
|---|---|---|---|---|---|
| Intercept | −0.350702 | 0.137087 | 128.928 | −2.558 | 0.012 |
| Time | −0.233797 | 0.121152 | 96.055 | −1.93 | 0.057 |
| CSF Aβ₁₋ | 0.085454 | 0.269908 | 132.122 | 0.317 | 0.752 |
| CSF | −8.95 × 10⁻⁵ | 0.000359 | 128.26 | −0.249 | 0.804 |
| CSF | −0.000954 | 0.000307 | 87.447 | −3.103 | 0.003 |
| Intercept | −0.325414 | 0.127507 | 130.073 | −2.552 | 0.012 |
| Time | −0.257617 | 0.118456 | 98.676 | −2.175 | 0.032 |
| CSF | −1.259219 | 0.275932 | 130.946 | −4.564 | 1.1 × 10⁻⁵ |
| CSF | −2.06 × 10⁻⁴ | 0.000336 | 129.619 | −0.613 | 0.541 |
| CSF | −0.000955 | 0.000302 | 90.817 | −3.159 | 0.002 |

CSF sPDGFRβ Predicting Change in Global Composite
Controlling for CSF Aβ₁₋₄₂ and pTau status

| | | | | | |
|---|---|---|---|---|---|
| Intercept | −0.238899 | 0.070962 | 140.235 | −3.367 | 0.001 |
| Time | −0.077554 | 0.044723 | 135.214 | −1.734 | 0.085 |
| CSF Aβ1- | 0.071522 | 0.145093 | 140.405 | 0.493 | 0.623 |
| CSF | −0.000278 | 0.000192 | 139.208 | −1.446 | 0.15 |
| CSF | −0.000304 | 0.000119 | 127.458 | −2.544 | 0.012 |
| Intercept | −0.234876 | 0.068014 | 139.987 | −3.453 | 0.001 |
| Time | −0.088201 | 0.043783 | 136.92 | −2.015 | 0.046 |
| CSF | −0.498812 | 0.154003 | 140.05 | −3.239 | 0.001 |
| CSF | −0.000297 | 0.000185 | 138.916 | −1.602 | 0.111 |
| CSF | −0.000313 | 0.000117 | 129.855 | −2.665 | 0.009 |

When stratified by APOE status, higher baseline CSF sPDGFRβ levels in APOE4 carriers predicted cognitive decline after controlling for CSF Aβ and pTau status, as shown in FIGS. 3d and 3e; and Table 6, but did not predict decline in APOE3 homozygotes, as shown in FIGS. 3f and 3g; and Table 7. FIGS. 3d and 3e illustrate that higher CSF sPDGFRβ (dashed line) in APOE4 carriers (n=58) significantly predicts future decline in mental status exam scores (p=0.005) after controlling for CSF Aβ (p=0.004) and pTau (p=0.003) status; (d), and in global cognitive composite scores (p=0.02) after controlling for CSF Aβ (p=0.02) and pTau (p=0.01) status (e). FIGS. 3f and 3g illustrate that baseline CSF sPDGFRβ does not predict decline (n=88) in either mental status (f) or global composite (g) scores in APOE3 homozygotes regardless of CSF Aβ or pTau status. In FIG. 3b-g, Separate lines indicate median split of baseline CSF sPDGFRβ (solid line, below median; dashed line, above median). Δ slopes provided for median split of baseline CSF sPDGFRβ groups. t0=−1 to 0.5 years post-LP, t1=0.5 to 2.5 years post-LP, and t2=2.5 to 4.5 years post-LP. Error bars show s.e. of the estimate. Linear mixed model analysis with no multiple comparison.

TABLE 6
Linear mixed model analysis of CSF sPDGFRβ baseline values
predicting future cognitive decline on age-, sex-, and education-
corrected z-scores on mental status exam and the global cognitive
composite of all neuropsychological tests in APOE4 carriers
after controlling for CSF Aβ and tau status. Significance
by linear mixed model analysis; no multiple comparison correction
applied. All tests are two-tailed (see Methods for further
details). Total sample of APOE4 carriers (n = 58).

| | | | | |
|---|---|---|---|---|
| β | SE | df | t | p-value |

CSF sPDGFRβ Predicting Change in Mental Status
Controlling for CSF Aβ₁₋₄₂

| | | | | | |
|---|---|---|---|---|---|
| Intercept | −0.493185 | 0.21685 | 53.123 | −2.274 | 0.027 |
| Time | −0.066464 | 0.229312 | 54.021 | −0.29 | 0.773 |
| CSF Aβ₁₋₄₂ | 0.209097 | 0.400371 | 54.583 | 0.522 | 0.604 |
| CSF sPDGFRβ | 0.000334 | 0.000546 | 52.841 | 0.612 | 0.543 |
| CSF sPDGFRβ | −0.001621 | 0.000542 | 45.708 | −2.993 | 0.004 |
| Intercept | −0.349128 | 0.199119 | 54.509 | −1.753 | 0.085 |
| Time | −0.127275 | 0.222438 | 55.358 | −0.572 | 0.57 |
| CSF pTau | −1.313143 | 0.399477 | 54.433 | −3.287 | 0.002 |
| CSF sPDGFRβ | 3.39 × 10⁻⁵ | 0.000503 | 53.885 | 0.067 | 0.946 |
| CSF sPDGFRβ | −0.001616 | 0.000525 | 47.055 | −3.077 | 0.003 |

CSF sPDGFRβ Predicting Change in Global Composite
Controlling for CSF Aβ₁₋₄₂ and pTau status

| | | | | | |
|---|---|---|---|---|---|
| Intercept | −0.334356 | 0.103951 | 53.211 | −3.216 | 0.002 |
| Time | −0.104365 | 0.071676 | 47.613 | −1.456 | 0.152 |
| CSF Aβ₁₋₄₂ | 0.126515 | 0.194343 | 45.506 | 0.651 | 0.518 |
| CSF sPDGFRβ | −0.000118 | 0.000263 | 53.224 | −0.449 | 0.655 |
| CSF sPDGFRβ | −0.00042 | 0.000168 | 39.136 | −2.502 | 0.017 |
| Intercept | −0.297598 | 0.099654 | 53.767 | −2.986 | 0.004 |
| Time | −0.113505 | 0.06901 | 50.395 | −1.645 | 0.106 |
| CSF pTau | −0.323346 | 0.198942 | 43.959 | −1.625 | 0.111 |
| CSF sPDGFRβ | −0.000147 | 0.000253 | 53.64 | −0.58 | 0.564 |
| CSF sPDGFRβ | −0.000434 | 0.000162 | 42.223 | −2.679 | 0.01 |

TABLE 7
Linear mixed model analysis of the overall incremental predictive
value of CSF sPDGFRβ baseline values in relation to cognitive
decline on age-, sex-, and education-corrected z-scores on mental
status exam and the global cognitive composite of all neuropsychological
tests in APOE3 carriers after controlling for CSF Aβ and tau
status. Significance by linear mixed model analysis; no multiple
comparison correction applied. All tests are two-tailed (see Methods
for further details). Total sample of APOE3 carriers (n = 88).

| | | | | |
|---|---|---|---|---|
| β | SE | df | t | p-value |

CSF sPDGFRβ Not Predicting Change in Mental Status
Controlling for CSF Aβ₁₋₄₂ and pTau

| | | | | | |
|---|---|---|---|---|---|
| Intercept | −0.351175 | 0.183267 | 366.785 | −1.916 | 0.056 |
| Time | −0.119878 | 0.145479 | 112.947 | −0.824 | 0.412 |
| CSF Aβ₁₋₄₂ status | −0.037947 | 0.36085 | 272.065 | −0.105 | 0.916 |
| CSF sPDGFRβ | −0.000446 | 0.000497 | 369.322 | −0.897 | 0.37 |
| CSF sPDGFRβ × | −0.000264 | 0.000402 | 111.691 | −0.658 | 0.512 |
| time | | | | | |
| Intercept | −0.380945 | 0.171377 | 306.273 | −2.223 | 0.027 |
| Time | −0.125378 | 0.142834 | 119.044 | −0.878 | 0.382 |
| CSF pTau status | −1.236054 | 0.375561 | 223.335 | −3.291 | 0.001 |
| CSF sPDGFRβ | −0.000478 | 0.000467 | 307.686 | −1.024 | 0.307 |
| CSF sPDGFRβ × | −0.00023 | 0.000399 | 117.444 | −0.577 | 0.565 |
| time | | | | | |

CSF sPDGFRβ Not Predicting Change in Global Composite
Controlling for CSF Aβ₁₋₄₂ and

| | | | | | |
|---|---|---|---|---|---|
| Intercept | −0.191169 | 0.09844 | 85.805 | −1.942 | 0.055 |
| Time | −0.048517 | 0.060892 | 90.359 | −0.797 | 0.428 |
| CSF Aβ₁₋₄₂ status | 0.028411 | 0.197739 | 86.711 | 0.144 | 0.886 |
| CSF sPDGFRβ | −0.000344 | 0.000281 | 85.181 | −1.223 | 0.225 |
| CSF sPDGFRβ × | −0.000176 | 0.000178 | 93.73 | −0.989 | 0.325 |
| time | | | | | |
| Intercept | −0.209294 | 0.094928 | 85.528 | −2.205 | 0.03 |
| Time | −0.054147 | 0.060094 | 90.311 | −0.901 | 0.37 |
| CSF pTau status | −0.50794 | 0.215262 | 86.808 | −2.36 | 0.021 |
| CSF sPDGFRβ | −0.000356 | 0.000272 | 84.783 | −1.311 | 0.193 |
| CSF sPDGFRβ × | −0.000165 | 0.000177 | 94.172 | −0.933 | 0.353 |
| time | | | | | |

Thus, high baseline levels of the BBB pericyte injury biomarker sPDGFRb in the CSF predicted future cognitive decline in APOE4 carriers, but not non-carriers, even after controlling for amyloid-b and tau status.

The increase in CSF sPDGFRβ with cognitive impairment was also found on cross-sectional CDR analysis in APOE4 carriers but not APOE3 homozygotes, as shown in FIGS. 4a and 4b, and Table 8. FIG. 4a illustrates CSF sPDGFRβ levels in CDR 0 APOE3 homozygotes (APOE3) (n=152) and APOE4 carriers (APOE4) (n=95) and with CDR 0.5 bearing APOE3 (n=42) or APOE4 (n=45). FIG. 4b illustrates CSF sPDGFRβ levels (estimated marginal means±s.e.m. from ANCOVA models corrected for age, sex, education, CSF Aβ₁₋₄₂ and pTau status) in individuals with CDR 0 bearing APOE3 (n=152) or APOE4 (n=95) and with CDR 0.5 APOE3 (n=42) and APOE4 (n=45).

TABLE 8
Hierarchical logistic regression analyses of CSF sPDGFRβ
baseline values predicting cognitive impairment in APOE4 but
not in APOE3 carriers based on clinical dementia rating (CDR) score
0.5 versus 0 after controlling for age, sex, education, HC and PHG
volumes, and CSF Aβ₁₋₄₂ and pTau status.

| | APOE4 carriers (n = 58) | | | | |
|---|---|---|---|---|---|
| CSF sPDGFRβ | | | | | |
| predicting CDR status | | | | | |
| | Model | −2 Log | Chi- | | |
| | Parameters | Likelihood | square | df | p-value |
| | for Step 1 | 122.370 | 6.582 | 1 | 0.01 |
| Step | Predictor | β | SE | Wald | p-value |
| 0 | Age (yrs) | 0.037 | 0.026 | 2.062 | 0.151 |
| 0 | Sex (ratio) | 0.57 | 0.459 | 1.543 | 0.214 |
| 0 | Education | −0.006 | 0.18 | 0.001 | 0.974 |
| | (attainment) | | | | |
| 0 | CSF Aβ₁₋₄₂ | −0.902 | 0.451 | 4.002 | 0.045 |
| | (status) | | | | |
| 0 | CSF pTau | −0.975 | 0.492 | 3.928 | 0.047 |
| | (status) | | | | |
| 1 | CSF | 0.001 | 0.001 | 6.127 | 0.013 |
| | sPDGFRβ | | | | |
| | (ng/mL) | | | | |

APOE3 carriers (n = 88)

| CSF sPDGFRβ | | | | | |
|---|---|---|---|---|---|
| predicting CDR status | | | | | |
| | Model | −2 Log | Chi- | | |
| | Parameters | Likelihood | square | df | p-value |
| | for Step 1 | 166.319 | 0.076 | 1 | 0.78 |
| Step | Predictor | β | SE | Wald | p-value |
| 0 | Age (yrs) | 0.069 | 0.024 | 8.472 | 0.004 |
| 0 | Sex (ratio) | 1.105 | 0.411 | 7.215 | 0.007 |
| 0 | Education | −0.273 | 0.158 | 3 | 0.083 |
| | (attainment) | | | | |
| 0 | CSF Aβ₁₋₄₂ | 0.106 | 0.418 | 0.065 | 0.799 |
| | (status) | | | | |
| 0 | CSF pTau | −0.675 | 0.433 | 2.433 | 0.119 |
| | (status) | | | | |
| 1 | CSF | 1.0 × 10⁻⁴ | 0.001 | 0.077 | 0.782 |
| | sPDGFRβ | | | | |
| | (ng/mL) | | | | |

Increased levels of sPDGFRβ in the CSF of APOE4 carriers correlated with increases in BBB permeability in the HC and PHG, as shown in FIGS. 4c and 4d and elevated levels of molecular biomarkers of BBB breakdown including albumin CSF/plasma quotient, and CSF fibrinogen and plasminogen, as shown in FIGS. 4e-4g.

FIGS. 4c and 4d illustrate the correlation between CSF sPDGFRβ and BBB Ktrans in the hippocampus (HC, n=65; c) and parahippocampal gyrus (PHG, n=65; d) in APOE4 carriers. FIGS. 4e-4g illustrate correlations between CSF sPDGFRβ and albumin quotient (Qalb, n=92; e), fibrinogen (n=93; f), and plasminogen (n=57; g) in APOE4 carriers.

Next, the proinflammatory cyclophilin A-matrix metalloproteinase-9 (CypA-MMP9) pathway was assessed. When activated by brain capillary pericytes in APOE4 (but not APOE3) knock-in mice, this pathway leads to MMP9-mediated breakdown of the BBB, which in turn induces neuronal stress related to leaked blood-derived neurotoxic proteins followed by neuronal dysfunction and loss of synaptic proteins. Brain tissue analysis has also shown higher activation of the CypA-MMP9 pathway in degenerating brain capillary pericytes in APOE4 carriers than in APOE3 homozygotes. In the cohort, APOE4 carriers, but not APOE3 homozygotes, developed an increase in CypA CSF levels with cognitive impairment, as shown in FIGS. 4h and 4i, which correlated with elevated CSF sPDGFRβ, as shown in FIG. 4j. FIG. 4h illustrates CSF cyclophilin A (CypA) in CDR 0 bearing APOE3 (n=75) and APOE4 (n=62) and with CDR 0.5 bearing APOE3 (n=33) or APOE4 (n=45) carriers. FIG. 4i illustrates CSF CypA levels (estimated marginal means±SEM from ANCOVA models corrected for age, sex, education, CSF Aβ₁₋₄₂ and pTau status) in CDR 0 APOE3 (n=75) and APOE4 (n=62) and with CDR 0.5 bearing APOE3 (n=33) or APOE4 (n=45). FIG. 4j illustrates the correlation between CSF CypA and sPDGFRβ in APOE4 carriers (n=96). APOE4 carriers, but not APOE3 homozygotes, also developed elevated MMP9 in the CSF with cognitive impairment, as shown in FIG. 4k, which correlated with elevated CSF CypA levels, as shown in FIG. 4l, suggesting that activation of the CypA-MMP9 pathway in APOE4 carriers correlates with pericyte injury, as shown in animal models. FIG. 4k illustrates CSF matrix metalloproteinase-9 (MMP9) in CDR 0 bearing APOE3 (n=72) and APOE4 (n=68) and CDR 0.5 bearing APOE3 (n=33) or APOE4 (n=45). FIG. 4l illustrates the correlation between CSF MMP9 and CypA in APOE4 carriers (n=104).

Thus, high baseline levels of the BBB pericyte injury biomarker sPDGFRβ in the CSF predicting future cognitive decline in APOE4 carriers, but not non-carriers, were correlated with increased activity of BBB-degrading cyclophilin A-matrix metalloproteinase 9 pathway in CSF.

There were no differences in glia or in inflammatory or endothelial cell injury CSF biomarkers between cognitively impaired and unimpaired APOE4 and APOE3 participants, but there was an increase in neuron-specific enolase (NSE) with cognitive impairment in APOE4 carriers, confirming neuronal stress and consistent with atrophy of the HC and PHG.

Together, these findings support the idea that the A β and tau pathways operate independently of the BBB breakdown pathway during the early stages of cognitive impairment in APOE4 carriers. In summary, the results show that BBB breakdown contributes to cognitive decline in APOE4 carriers independent of AD pathology; that high baseline CSF levels of sPDGFRβ can predict future cognitive decline in APOE4 carriers; and that APOE4, but not APOE3, activates the CypA-MMP9 pathway in the CSF, which may lead to accelerated BBB breakdown and thereby cause neuronal and synaptic dysfunction. As blockade of the CypA-MMP9 pathway in APOE4 knock-in mice restores BBB integrity and subsequently normalizes neuronal and synaptic function19, it is possible that CypA inhibitors (some of which have been used in humans for non-neurological applications 31) might also suppress the CypA pathway in cerebral blood vessels in APO4 carriers.

In the detailed description, references to “various embodiments”, “one embodiment”, “an embodiment”, “an example embodiment”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. After reading the description, it will be apparent to one skilled in the relevant art(s) how to implement the disclosure in alternative embodiments.

Benefits, other advantages, and solutions to problems have been described herein with regard to specific embodiments. However, the benefits, advantages, solutions to problems, and any elements that may cause any benefit, advantage, or solution to occur or become more pronounced are not to be construed as critical, required, or essential features or elements of the disclosure. The scope of the disclosure is accordingly to be limited by nothing other than the appended claims, in which reference to an element in the singular is not intended to mean “one and only one” unless explicitly so stated, but rather “one or more.” Moreover, where a phrase similar to ‘at least one of A, B, and C’ or ‘at least one of A, B, or C’ is used in the claims or specification, it is intended that the phrase be interpreted to mean that A alone may be present in an embodiment, B alone may be present in an embodiment, C alone may be present in an embodiment, or that any combination of the elements A, B and C may be present in a single embodiment; for example, A and B, A and C, B and C, or A and B and C.

All structural, chemical, and functional equivalents to the elements of the above-described various embodiments that are known to those of ordinary skill in the art are expressly incorporated herein by reference and are intended to be encompassed by the present claims. Moreover, it is not necessary for an apparatus or component of an apparatus, or method in using an apparatus to address each and every problem sought to be solved by the present disclosure, for it to be encompassed by the present claims. Furthermore, no element, component, or method step in the present disclosure is intended to be dedicated to the public regardless of whether the element, component, or method step is explicitly recited in the claims. No claim element is intended to invoke 35 U.S.C. 112(f) unless the element is expressly recited using the phrase “means for.” As used herein, the terms “comprises”, “comprising”, or any other variation thereof, are intended to cover a non-exclusive inclusion, such that a chemical, chemical composition, process, method, article, or apparatus that comprises a list of elements does not include only those elements but may include other elements not expressly listed or inherent to such chemical, chemical composition, process, method, article, or apparatus.

We claim:

1. A method for determining a concentration of soluble platelet-derived growth factor β (sPDGFRβ) in a biofluid sample from a human subject, the method comprising:
forming a ternary complex of a detection antibody comprising a labelled anti-human PDGFRβ biotinylated antibody, sPDGFRβ present in the biofluid sample, and a capture antibody comprising an anti-human PDGFRβ antibody, wherein the anti-human PDGFRβ antibody is bound to a surface;
detecting an intensity of light emission from the ternary complex; and
interpolating the intensity of the light emission on a calibration curve to obtain the concentration of sPDGFRβ in the biofluid sample,
wherein the labelled anti-human PDGFRβ biotinylated antibody comprises a conjugate between an immunoassay detection reagent and the anti-human PDGFRβ biotinylated antibody.

2. The method of claim 1, wherein the capture antibody comprises a goat anti-human PDGFRβ polyclonal antibody.

3. The method of claim 1, wherein the detection antibody comprises a goat anti-human PDGFRβ biotinylated polyclonal antibody.

4. The method of claim 1, wherein the biofluid comprises human cerebrospinal fluid (CSF), blood serum or blood plasma.

5. The method of claim 1, wherein the concentration of sPDGFRβ in the biofluid sample is from about 100 pg/mL to about 30,000 pg/mL.

6. The method of claim 1, wherein the immunoassay detection reagent comprises a sulfur-tagged streptavidin reagent.

7. The method of claim 1, wherein the labelled anti-human PDGFRβ biotinylated antibody further comprises a streptavidin-biotin conjugated electrochemiluminescence label.

8. The method of claim 7, further comprising applying a voltage to the ternary complex during the detecting step.

9. The method of claim 8, wherein the surface comprises an electrode surface disposed in a well plate.

10. The method of claim 9, wherein the detecting step further comprises detection of an electrochemiluminescence intensity upon insertion of the well plate into an imager having electrochemiluminescence detection.

11. The method of claim 10, wherein the calibration curve comprises an x/y plot of electrochemiluminescence intensity versus sPDGFRβ concentration.

12. The method of claim 9, wherein the capture antibody is bound to a bottom of the well plate by spot-coating the bottom of the well plate with a phosphate buffered solution comprising a goat anti-human PDGFRβ polyclonal antibody and polysorbate 20.

13. The method of claim 12, wherein the ternary complex is formed in a two-step process consisting of: (a) exposing the bound goat anti-human PDGFRβ polyclonal antibody in the well plate to a diluted aliquot of the biofluid sample to form a binary complex of sPDGFRβ and the capture antibody; and (b) exposing the binary complex to a solution comprising a labelled goat anti-human PDGFRβ biotinylated polyclonal antibody.

14. The method of claim 1, wherein the presence of sPDGFRβ in the biofluid sample provides a pericyte injury biomarker indicative of brain microvascular and blood brain barrier (BBB) injury.

15. The method of claim 1, wherein the presence of sPDGFRβ in the biofluid sample indicates presence of at least one neurodegenerative disorder selected from Parkinson's Disease, Huntington's Disease, Human Immunodeficiency Virus (HIV)-dementia, or Post-Traumatic Brain Syndrome.

16. The method of claim 1, wherein the immunoassay detection reagent comprises horseradish peroxidase (HRP)-conjugated streptavidin.

17. The method of claim 16, wherein the calibration curve comprises an x/y plot of absorbance versus sPDGFRβ concentration.

18. A method of determining the presence of cognitive impairment or dementia in a human subject, the method comprising obtaining a concentration of sPDGFRβ in a biofluid sample obtained from the human subject according to the method of claim 1, wherein the subject is categorized as having cognitive impairment or dementia if the sPDGFRβ in the biofluid sample is greater than about 4,000 pg/mL.

19. The method of claim 18, wherein the human subject is categorized as having dementia if the sPDGFRβ in the biofluid sample from the subject is greater than about 5,000 pg/mL.

20. A method of determining the presence of Alzheimer's disease in a human subject, the method comprising obtaining a concentration of sPDGFRβ in a biofluid sample obtained from the human subject according to the method of claim 1, wherein the subject is categorized as having Alzheimer's disease if the sPDGFRβ in the biofluid sample is greater than about 4,000 pg/mL.

21. The method of claim 20, wherein the human subject is categorized as having Alzheimer's disease if the sPDGFRβ in the biofluid sample from the subject is greater than about 5,000 pg/mL.

22. An assay system for determining a concentration of soluble platelet-derived growth factor β (sPDGFRβ) in a biofluid sample, the assay system comprising:
a ternary complex of a detection antibody comprising a labelled goat anti-human PDGFRβ biotinylated polyclonal antibody, sPDGFRβ present in the biofluid sample, and a capture antibody comprising a goat anti-human PDGFRβ polyclonal antibody, wherein the goat anti-human PDGFRβ polyclonal antibody is bound to a surface, and wherein the labelled goat anti-human PDGFRβ biotinylated antibody is a conjugation product of an immunoassay detection reagent and the goat anti-human PDGFRβ biotinylated polyclonal antibody.
